(19) United States
(12) Patent Application Publication
(10) Pub. No.: US 2024/0156829 A1
(43) Pub. Date: May 16, 2024

(54) Stable Antiemetic Emulsions for Parenteral Administration

(71) Applicant: Slayback Pharma LLC, Princeton, IN (US)

(72) Inventors: Ashish Anilrao Dubewar, Hyderabad (IN); Rahul Dhutaji Bhise, Hyderabad (IN); Shanker Mamidi, Nalgonda (IN); Mahadeo Vasant Mahadik, Hyderabad (IN); Sumitra Ashokkumar Pillai, Hyderabad (IN); Satheesh Balasubramanian, Hyderabad (IN); Kumar Swamy Ummiti, Hyderabad (IN); Mayur Anshiram Adhav, Hyderabad (IN); Praveen Kumar Subbappa, Princeton, NJ (US)

(21) Appl. No.: 18/487,330

(22) Filed: Oct. 16, 2023

(30) Foreign Application Priority Data
Oct. 31, 2022 (IN) 202241061823

(51) Int. Cl. A61K 31/5377; A61K 9/00; A61K 9/107; A61K 47/12; A61K 47/26; A61K 47/44

(52) U.S. Cl. CPC A61K 31/5377; A61K 9/0019; A61K 9/107; A61K 47/12; A61K 47/26; A61K 47/44

(57) ABSTRACT

The present invention relates to stable pharmaceutical compositions for parenteral administration comprising aprepitant. The present invention further relates to a method for manufacturing the composition as well as to the use of these compositions. Also included are stable pharmaceutical compositions for parenteral administration comprising aprepitant and palonosetron. The pharmaceutical compositions are stable oil-in-water emulsions for parenteral administration and are particularly useful for the prevention and control of acute and delayed chemotherapy-induced nausea and vomiting (CINV), for the prevention of postoperative nausea and vomiting (PONV), and/or for the prevention of radiation induced nausea and vomiting (RINV).

### CROSS REFERENCE TO RELATED APPLICATIONS

This application claims the benefit of Indian Provisional Application No. 202241061823, as filed on Oct. 31, 2022, the entire contents of which are incorporated herein.

### FIELD OF THE INVENTION

The present invention relates to stable pharmaceutical compositions for parenteral administration comprising aprepitant. The present invention further relates to a method for manufacturing the inventive composition as well as to the use of these inventive compositions. Also included are stable pharmaceutical compositions for parenteral administration comprising aprepitant and palonosetron. The inventive pharmaceutical compositions are stable oil-in-water emulsions for parenteral administration and are particularly useful for the prevention and control of acute and delayed chemotherapy-induced nausea and vomiting (CINV), for the prevention of postoperative nausea and vomiting (PONV) and/or for the prevention of radiation induced nausea and vomiting (RINV).

### BACKGROUND OF THE INVENTION

Aprepitant (5-([(2R, 3S)-2-((R)-1-[3,5-bis (trifluoromethyl) phenyl] ethoxy)-3-(4-fluoro-phenyl) morpholino]methyl)-1H-1,2,4-triazol-3(2H)-one) is an antiemetic compound that belongs to the class of substance P/neurokinin 1 (NK-1) receptor antagonists that mediate their effect by blocking the NK-1 receptor. U.S. Pat. No. 6,297,375 suggests that NK-1 antagonists, such as aprepitant, are useful for treating a variety of conditions in which substance P (the natural ligand for the NK-1 receptor) is active,

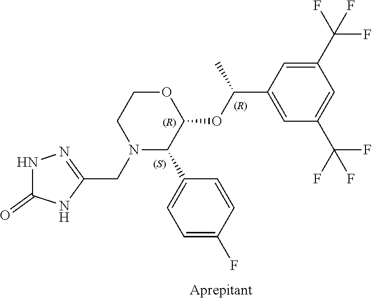

Currently, aprepitant is marketed in the United States under brand names Emend® (oral capsules; 40 mg, 80 mg & 125 mg and powder for oral suspension; 125 mg/kit); Cinvanti® (IV emulsion; 130 mg/18 mL) and Aponvie® (IV emulsion; 32 mg/4.4 mL). Emend® has been indicated in combination with other antiemetic agents, in pediatrics and adult population for prevention of acute and delayed nausea and vomiting associated with initial and repeat courses of highly emetogenic cancer chemotherapy (HEC) and moderately emetogenic cancer chemotherapy (MEC). Cinvanti®, in addition to above, is indicated for the prevention of nausea and vomiting associated with initial and repeat courses of MEC as a 3-day regimen. However, the use of Cinvanti®, is confined to use only in adult populations. Aponvie is indicated for the prevention of postoperative nausea and vomiting (PONV) in adults.

Nausea describes the unpleasant sensation of an imminent need to vomit, whereas vomiting refers to the forceful oral expulsion of gastric contents associated with contraction of the abdominal and chest wall musculature. In such circumstances, oral administration of drugs is not a feasible option, as there is a likely possibility of expulsion of orally administered drug (either solid or as a liquid), thus resulting in delivery of incomplete and inaccurate dosages of the active compound. Hence, during the management of CINV. PONV and/or RINV, it is desirable to formulate aprepitant suitable for parenteral or intravenous administration.

Aprepitant however, is having very poor solubility, and thus it is very challenging to formulate aqueous parenteral formulations of aprepitant for purposes of long-term storage and for administration. An effective means of addressing this challenge, is to prepare an emulsion which may allow preparation of an injectable formulation with a high drug loading.

As noted above, commercial emulsion formulations of aprepitant are available in US under the brand names Cinvanti® and Aponvie®. These injectable emulsions of aprepitant can be administered intravenously as an infusion and also as a bolus preparation. According to their approved labels, both these formulation vials must be stored at refrigerated conditions of 2-8° C. and are stable at room temperature for only up to 60 days. Additionally, U.S. patent publication no. 2022/0160722 (“Steps Biosciences”) seeks to formulate injectable aprepitant emulsion formulations having better stability at room temperature in comparison with the commercially approved injectable emulsion formulations of aprepitant. A more stable and robust form of aprepitant parenteral emulsion which can be formulated and maintained stable at room temperature for prolonged period of time, is a current need.

Furthermore, NK-1 receptor antagonists such as, Aprepitant or Netupitant are indicated for the prevention of emesis, in combination with 5HT3 antagonists and/or steroids. 5HT3 antagonists, such as palonosetron hydrochloride [(3aS)-2-[(3S)-1-azabicyclo [2.2.2]octan-3-yl]-3a,4,5,6-tetrahydro-3H-benzo[de] isoquinolin-1-one; hydrochloride], has emerged as an excellent tool for combating nausea and vomiting from emetogenic medical procedures, including chemotherapy (CINV), surgery (PONV) and radiation therapy (RINV). Palonosetron hydrochloride is considered to be more potent in comparison to most of the existing 5HT3 antagonists and it has a half-life of 40 hours, which effectively combats delayed onset nausea or vomiting.

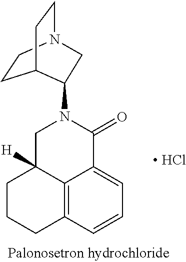 [SMILES: O=C1c2cccc3c2[C@H](CCC3)CN1[C@@H]1CN2CCC1CC2 ; "· HCl"]

There exists a need for developing a single combined dose of aprepitant and palonosetron hydrochloride, for the prevention of nausea and vomiting during the acute and delayed phases of CINV, PONV and/or RINV. However, formulating palonosetron in liquid formulations has not proven to be an easy task, because it is easily oxidized and degraded by light or oxygen or heavy metals in a liquid medium.

International Application no. PCT/EP2004/000888 discloses shelf-stable formulations of palonosetron for reducing emesis, wherein formulations are rendered stable using a tonicifying agent and/or a chelating agent and by adjusting and maintaining the pH between 4.0 to 6.0 using a buffering agent. Although, palonosetron can be stabilized in a liquid medium, the fate of a combined formulation of aprepitant and palonosetron in a liquid medium is unpredictable. Accordingly, there is a long-standing need in the art to provide a liquid formulation suitable for parenteral administration comprising aprepitant and palonosetron, exhibiting a prolonged storage stability.

In view of the above, there exists a need for developing improved formulations for providing effective therapy for treating or preventing nausea and vomiting particularly emanating from chemotherapy, radiotherapy and surgery. Because of the problems associated with commercially approved dosage forms of aprepitant, it is always desirable to develop improved parenteral emulsions of aprepitant, which are therapeutically effective, and exhibit prolonged room temperature stability without any significant loss of potency, enabling optimal usage of aprepitant compositions. Additionally, in order to improve patient compliance and, more importantly, in order to accentuate the anti-emetic effect, particularly in the delayed phases of CINV, PONV and/or RINV, there exists a need of a novel, stable, parenteral emulsion of aprepitant and palonosetron.

The present invention fulfils such needs by developing stable emulsions for parenteral administration of aprepitant, in order to achieve an improved standard of patient care. Additionally, the present invention further provides stable emulsions for parenteral administration comprising aprepitant and palonosetron, in order to achieve an improved standard of patient care.

### SUMMARY OF THE INVENTION

In one aspect, the present invention relates to the use of inventive formulations comprising centrally acting NK1 antagonists, particularly aprepitant, for the prevention and control of acute and delayed chemotherapy-induced nausea and vomiting (CINV), for the prevention of postoperative nausea and vomiting (PONV) and/or for the prevention of radiation induced nausea and vomiting (RINV).

The present invention relates to stable aprepitant emulsions for parenteral administration, suitable for human use, with prolonged room temperature stability compared to the currently approved and marketed dosage forms of aprepitant.

The inventive pharmaceutical composition is suitable for parenteral administration via subcutaneous, intravenous or intramuscular routes and is provided in the form of an emulsion.

The inventive injectable pharmaceutical compositions are advantageously ready-to-use (RTU) or ready-to-dilute (RTD). An aspect of the invention relates to stable ready-to-use or ready-to-dilute aprepitant compositions suitable for parenteral administration.

The present invention relates to parenteral emulsion formulations of aprepitant, particularly wherein aprepitant is present at a concentration ranging from about 2 mg/mL to about 20 mg/mL, preferably about 7.2 mg/mL.

In one aspect, a stable emulsion suitable for parenteral administration is provided comprising an oil phase, wherein the oil phase comprises aprepitant, an emulsifier, a co-emulsifier and an oil; and an aqueous phase, wherein the aqueous phase comprises water, a tonicity adjuster and buffering agent.

In another aspect, a stable emulsion suitable for parenteral administration may further optionally comprise one or more pharmaceutically acceptable excipients selected from the group consisting of surfactants, co-surfactants, co-solvents, solubilizers, oils, pH adjusting agents, antioxidants, chelating agents and preservatives.

In an aspect, the ratio of oil phase to aqueous phase (wt %:wt %) in the composition ranges from about 20:80 to about 80:20, about 25:75 to about 75:25 or about 30:70 to about 70:30 or about 35:65 to about 65:35 or about 40:60 to about 60:40. In another embodiment, the ratio of oil phase to aqueous phase (wt %:wt %) in the composition is about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61 or about 40:60. In a more preferred embodiment, the ratio of oil phase to aqueous phase (wt %:wt %) in the composition is about 30:70.

In an aspect, the ratio of oil phase to aqueous phase (wt %:wt %) ranges from about 30:70 to about 60:40, about 35:65 to about 50:50, about 40:60 to about 45:55, about 33:67 to about 53:47. In another embodiment, the ratio of oil phase to aqueous phase (wt %:wt %) is about 25:75, about 30:70, about 35:75, about 40:60, about 45:55, about 50:50, about 55:45, about 60:40, about 65:35. In a more preferred embodiment, the ratio of oil phase to aqueous phase (wt %:wt %) is about 45:55.

In one aspect, the composition comprises about 10 wt/wt % (weight/weight %) to about 25 wt/wt %, about 12 wt/wt % to about 22 wt/wt %, about 15 wt/wt % to about 20 wt/wt %, preferably about 17 wt/wt % to about 18 w/wt % of emulsifier. In another embodiment, the composition comprises about 15 wt/wt %, about 16 wt/wt %, about 17 wt/wt %, about 18 wt/wt %, about 19 wt/wt %, about 20 wt/wt %, about 21 wt/wt %, about 22 wt/wt %, about 23 w/wt %, about 24 wt/wt % or about 25 wt/wt % of emulsifier. In another aspect, the emulsifier is lecithin. In yet another aspect, the lecithin is egg yolk lecithin also called as egg lecithin.

In an aspect, the ratio of emulsifier to aprepitant (wt %:wt %) in the composition ranges from about 15:1 to about 40:1, about 20:1 to about 30:1 or about 22:1 to about 27:1. In another aspect, the ratio of emulsifier to aprepitant (wt %:wt %) in the composition is about 20:1, about 21:1, about 22:1, about 23:1, about 24:1, about 25:1 about 26:1, about 27:1, about 28:1, about 29:1 or about 30:1.

In an aspect, the inventive composition has a pH of about 6 to about 12, about 6 to about 11, about 6 to about 10, about 6 to about 9, about 7 to about 9, about 7 to about 11, about 7.5 to about 11, about 7.5 to about 8.7, about 7.5 to about 9.

In an aspect, the composition is a stable fine emulsion maintaining an intensity-weighted mean particle size as determined by dynamic light scattering (DLS) of about 30 nm to about 250 nm. In another embodiment, the average droplet size is maintained below about 150 nm for a period of at least 1 month, 3 months, 6 months, 9 months, 12 months, 2 years or 3 years at 5° C. or at room temperature.

In an aspect, a method for preparing an aprepitant emulsion suitable for parenteral administration is provided, the method comprising: a) preparing an oil phase by dissolving an emulsifier in a co-emulsifier to provide an emulsifier mixture, followed by dissolving aprepitant in the emulsifier mixture and adding an oil to generate an oil-based mixture; b) preparing an aqueous phase by mixing water with tonicity adjuster, buffering agent and optionally an osmotic agent and/or a pH modifier to generate an aqueous mixture; c) combining the oil-based mixture and the aqueous mixture to provide a combined mixture and subjecting the combined mixture to high-speed homogenization to generate a crude emulsion; and d) subjecting the crude emulsion to high pressure homogenization to generate a fine emulsion.

In one aspect, the method further comprises sterilizing the fine emulsion to generate the final emulsion, wherein the final emulsion is suitable for injection into a human subject. The inventive injectable emulsion may be sterilized using a technique selected from the group consisting of filtration through aseptic filtration-filling-sealing process, terminal sterilization, autoclaving, incorporation of sterilizing agents, irradiation, and heating.

In an aspect, the oil phase and aqueous phase are prepared at a temperature of about 50° C. to about 60° C., more preferably at about 55° C. In another aspect, the high-speed homogenization is performed at about 50° C. to about 60° C., more preferably at about 55° C. In yet another aspect, the high-pressure homogenization is performed with cooling, which is sufficient to bring the temperature of the emulsion at the outlet of the process to about 25° C. or about 30° C. within the time period.

In a further aspect, the dissolution of emulsifier in the co-emulsifier is performed in a step-wise manner.

The inventive injectable emulsion may be disposed in a vial or a pre-filled syringe.

In an aspect, a method for preparing an aprepitant emulsion suitable for parenteral administration is provided, wherein the method comprises (a) combining an emulsifier and co-emulsifier in a sequential manner, followed by addition of aprepitant, followed by addition of an oil to generate an oil phase; (b) combining water, a tonicity adjuster and buffering agent to generate an aqueous phase; (c) homogenizing the oil phase with the aqueous phase to generate a pharmaceutical emulsion; and (d) sterilizing the pharmaceutical emulsion.

In an aspect, the process of preparation of a stable injectable emulsion composition of aprepitant is provided, wherein the process comprises; (a) combining an emulsifier and co-emulsifier in a sequential manner, followed by addition of aprepitant, followed by addition of an oil to generate an oil phase; (b) combining water, a tonicity adjuster and buffering agent to generate an aqueous phase; (c) homogenizing the oil phase with the aqueous phase to generate the injectable emulsion composition; and (d) sterilizing the injectable emulsion composition, wherein the preparation of oil phase and aqueous phase is carried out at a temperature of about 55° C.

In another aspect, a method for preparing aprepitant emulsion suitable for parenteral administration is provided, wherein the method comprises (a) combining egg lecithin and ethanol in a sequential manner, followed by addition of aprepitant, followed by addition of soybean oil to generate an oil phase; (b) combining water, sucrose and sodium oleate to generate an aqueous phase; (c) homogenizing the oil phase with the aqueous phase to generate the aprepitant emulsion; and (d) sterilizing the aprepitant emulsion.

In an aspect, a stable emulsion suitable for parenteral administration is provided comprising (i) an oil phase, which comprises aprepitant, emulsifier, a co-emulsifier and an oil; and (ii) an aqueous phase, which comprises water, a tonicity adjuster and buffering agent, wherein the oil phase is substantially free of buffering agent.

In an aspect, a method for preparing an aprepitant emulsion suitable for parenteral administration is provided, wherein the method comprises (i) preparing an oil phase comprising aprepitant, emulsifier, co-emulsifier and an oil; (ii) preparing an aqueous phase consisting of water, tonicity adjuster and buffering agent; (iii) homogenizing the oil phase with the aqueous phase to generate the aprepitant emulsion; and (iv) sterilizing the aprepitant emulsion.

In an aspect, a stable composition suitable for parenteral administration is provided comprising (i) an oil phase which comprises (a) aprepitant, (b) egg lecithin, (c) ethanol, and (d) soybean oil; and (ii) an aqueous phase which comprises (a) water, (b) sucrose, and (c) sodium oleate; wherein the ratio of egg lecithin to aprepitant (wt %:wt %) in the composition is about 25:1; wherein the egg lecithin concentration in the composition is about 18 wt/wt %; wherein the composition is an emulsion; wherein the ratio of the oil phase to the aqueous phase (wt %:wt %) in the composition is about 30:70; and wherein the composition remains stable at 25° C./60% RH for at least 6 months.

In an aspect, a method for preparing an aprepitant emulsion suitable for parenteral administration is provided, wherein the method comprises (i) preparing an oil phase comprising aprepitant, egg lecithin, ethanol, and soybean oil; (ii) preparing an aqueous phase consisting of water, sucrose and sodium oleate; (iii) homogenizing the oil phase with the aqueous phase to generate the aprepitant emulsion; and (iv) sterilizing the aprepitant emulsion.

In a further aspect, the injectable emulsion upon intravenous or intramuscular administration exhibits bioequivalence to a commercially available reference drug products (such as Cinvanti® and Aponvie®), wherein said bioequivalence is established by at least one of: (i) a confidence interval for mean AUC0-t between about 80% and about 125%; (ii) a confidence interval for mean AUC0-infinity between about 80% and about 125%; (iii) a confidence interval for mean Cmax between about 80% and about 125% or a combination thereof.

In one aspect, the injectable emulsion comprising aprepitant is physically stable, without any phase separation, for at least 60 days, when stored at 25° C./60% RH or at 2° C. to 8° C. or at room temperature.

In another aspect, the injectable emulsion comprising aprepitant is chemically stable, wherein the level of total impurities is less than about 3.0% w/w, for at least 60 days, when stored at 25° C./60% RH or at 2° C. to 8° C. or at room temperature

In yet another aspect, the injectable emulsion may exist in a 4.4 mL or 18 mL single-dose vial.

The present invention further relates to stable parenteral emulsion of aprepitant and palonosetron hydrochloride, wherein aprepitant is present at a concentration ranging from about 2 mg/mL to about 20 mg/mL, preferably about 7.2 mg/mL and palonosetron hydrochloride is present at concentration ranging from about 0.001 mg/mL to about 1 mg/mL, preferably about 0.0155 mg/mL (equivalent to 0.0138 mg of palonosetron, as free base).

In one aspect, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent, pH adjusting agents, osmotic agents and antioxidants, wherein aprepitant is present in oil phase and palonosetron is present either in oil phase or in aqueous phase.

In another aspect, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, a buffering agent, a pH adjusting agent, an osmotic agent and an antioxidant, wherein aprepitant is substantially present in the oil phase and palonosetron is substantially present either in the oil phase or in aqueous phase.

In another aspect, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, a buffering agent, a pH adjusting agent, an osmotic agent and an antioxidant, wherein aprepitant is substantially present in the oil phase and palonosetron is substantially present in aqueous phase.

In another aspect, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, a buffering agent, a pH adjusting agent, an osmotic agent and an antioxidant, wherein aprepitant and palonosetron are present in the oil phase.

In one aspect, a stable emulsion of aprepitant and palonosetron suitable for parenteral administration is provided comprising (i) an oil phase, which comprises an emulsifier, a co-emulsifier, an oil and an antioxidant; and (ii) an aqueous phase, which comprises water, a tonicity adjuster, a buffering agent, a pH adjusting agent, an osmotic agent and an antioxidant, wherein the antioxidant is present either in the oil phase or in aqueous phase.

In one aspect, a stable emulsion of aprepitant and palonosetron suitable for parenteral administration is provided comprising (i) an oil phase, which comprises an emulsifier, a co-emulsifier, an oil and an antioxidant; and (ii) an aqueous phase, which comprises water, a tonicity adjuster, a buffering agent, a pH adjusting agent, an osmotic agent and an antioxidant, wherein the antioxidant is substantially present either in the oil phase or in the aqueous phase.

In one aspect, a stable emulsion of aprepitant and palonosetron suitable for parenteral administration is provided comprising (i) an oil phase, which comprises an emulsifier, a co-emulsifier, an oil and an antioxidant; and (ii) an aqueous phase, which comprises water, a tonicity adjuster, a buffering agent, a pH adjusting agent, an osmotic agent and an antioxidant, wherein the antioxidant is substantially present in both oil phase as well as in the aqueous phase.

In an aspect, preparing the oil phase comprises dissolving palonosetron and aprepitant in a mixture of an emulsifier and co-emulsifier. In another aspect, preparing the aqueous phase comprises dissolving palonosetron in a mixture of water, tonicity adjuster, buffering agent and an antioxidant.

In one aspect, the injectable emulsion comprising aprepitant and palonosetron is physically stable, without any phase separation, when stored for at least 60 days at 25° C./60% RH or at 2° C. to 8° C. or at room temperature.

In one aspect, the injectable emulsion comprising aprepitant and palonosetron is chemically stable, wherein the level N-oxide impurity is less than about 0.1% w/w, when stored for at least 60 days at 25° C./(60% RH or at 2° C. to 8° C. or at room temperature.

In one aspect, the injectable emulsion comprising aprepitant and palonosetron is chemically stable, wherein the level total impurities impurity is less than about 3.0% w/w, when stored for at least 60 days at 25° C./60% RH or at 2° C. to 8° C. or at room temperature.

In one aspect, the composition is a stable injectable composition comprising (i) an oil phase which comprises (a) aprepitant, (b) egg lecithin, (c) ethanol, and (d) soybean oil; and (ii) an aqueous phase which comprises (a) water, (b) sucrose, and (c) sodium oleate; wherein the ratio of egg lecithin to aprepitant (wt %:wt %) in the composition is about 25:1; wherein the egg lecithin concentration in the composition is about 18 wt/wt %; wherein the composition is an emulsion; wherein the ratio of the oil phase to the aqueous phase (wt %:wt %) in the composition is about 30:70; and wherein the composition remains stable at 25° C./60% RH for at least 6 months.

### BRIEF DESCRIPTION OF THE DRAWINGS

FIG. 1 illustrates the manufacturing procedure flow chart.

### DETAILED DESCRIPTION OF THE INVENTION

Unless defined otherwise, all the technical and scientific terms used herein have the same meanings as commonly known by a person skilled in the art. In case there are a plurality of definitions for the terms herein, the definitions provided herein will prevail.

As used herein, “a,” “an” or “the” are used interchangeably. As used herein, the term “or” is generally employed in its usual sense including “and/or” unless the content clearly dictates otherwise.

As used herein, the term “aprepitant” refers to aprepitant free base or its pharmaceutically acceptable salt, solvate or hydrate thereof.

As used herein, the term “palonosetron” refers to palonosetron free base or its pharmaceutically acceptable salt, solvate or hydrate thereof.

As used herein, the term “about” means having a value falling within an accepted standard of error of the mean when considered by one of ordinary skill in the art. Frequently, the term “about” refers to ±10%, preferably ±5% of the value or range to which it refers. However, when the term “about” is used in connection with pH, it should be considered as ±2 units of the pH value.

Within the context of the present invention, the term “ready-to-use” or “RTU” as used herein refers to an injectable composition that is stable and is not lyophilized before being reconstituted. The term “ready-to-use” or “RTU” also encompasses within its scope, injectable compositions that are stable and do not require any reconstitution or dilution with parenterally acceptable diluent and can be directly administered to the patient.

Within the context of the present invention, the term “ready-to-dilute” or “RTD” as used herein refers to an injectable composition that is stable and can be diluted with a suitable diluent for parenteral administration.

The terms “pharmaceutical composition”, “pharmaceutical product”, “dosage form”, “pharmaceutical dosage form” “formulation”, “pharmaceutical formulation”, etc., refer to a composition that may be administered to a patient in need of treatment. For example, in one aspect, the term “pharmaceutical composition,” as used herein, refers to an emulsion for parenteral administration.

The term “emulsion” or “emulsion formulation” means a colloidal dispersion of two immiscible liquids in the form of droplets, whose diameter, in general, is between about 10 nanometers and about 100 microns. An emulsion is denoted by the symbol O/W (oil-in-water) if the continuous phase is an aqueous solution and by W/O (water-in-oil) if the continuous phase is an oil. Oil-in-water emulsions for parenteral administration have to be sterile, pyrogen-free, well tolerated, isotonic or as close as possible to isotonicity, free of particulate impurities and storage stable. Their pH should be as close as possible to the pH of the blood.

The term “pharmaceutically acceptable excipient” as used herein means a diluent, carrier, or composition auxiliary, which is non-toxic and inert, which does not have undesirable effects on a subject to whom it is administered and is suitable for delivering a therapeutically active agent to the target site without affecting the therapeutic activity of the active agent.

The term “substantially” means, e.g., not entirely complete, or not entirely absolute. Typically, “substantially” should be understood to refer to at least about 90 percent. For example. “substantially” can mean at least about 95 percent, at least about 96 percent, at least about 97 percent, at least about 98 percent, or at least about 99 percent. In a preferred embodiment, “substantially” refers to at least about 99.5 percent. In a more preferred embodiment, “substantially” refers to at least about 99.9 percent.

Intravenous emulsions should have a very small droplet size to circulate in the bloodstream without causing capillary blockage and embolization. These size limits are typified by USP33-NF28 General Chapter <729> for Globule Size Distribution in Lipid Injectable Emulsions, hereinafter referred to as USP <729>, which defines universal limits for (1) mean droplet size not exceeding 500 nm or 0.5 μm and (2) the population of large-diameter fat globules, expressed as the volume-weighted percentage of fat greater than 5 μm (PFAT5) not exceeding 0.05%, irrespective of the final lipid concentration. The droplet size limits defined in USP <729> apply throughout the assigned shelf life.

The terms “stable” and “stability” mean that the evolution of the product with time and/or under specific environmental conditions (i.e., temperature, humidity, etc.) has no significant effects on its quality, safety and/or efficacy for a given time period. It can be measured through formation of degradation products (impurities), variation of pH, appearance (e.g., precipitation), microbial growth, and/or color. The term “stable” indicates both physical stability as well as chemical stability.

The term “physically stable” mean emulsions will meet the criteria under following: (1) meets the criteria for mean droplet size not exceeding 500 nm or 0.5 μm, as per USP <729>; (2) meets the criteria for ‘the population of large-diameter fat globules’ (as per USP <729>), expressed as the volume-weighted percentage of fat greater than 5 μm (PFAT5) not exceeding 0.05%, at 5° C. or room temperature for a time period equal to or at least 1 week, 2 weeks, 1 month, 2 months, 6 months, 1 year or 2 years; (3) will have no visible aprepitant crystals upon storage at 5° C. or room temperature for a time period equal to or at least 1 week, 2 weeks, 1 month, 2 months, 6 months, 1 year or 2 years; (4) will have no tendency of phase separation between the oil phase and the aqueous phase, upon storage at 5° C. or room temperature for a time period equal to or at least 1 week, 2 weeks, 1 month, 2 months, 6 months, 1 year or 2 years.

The term “chemically stable” means that no more than about 10% loss of aprepitant under typical commercial storage conditions. Preferably, formulations of the present invention will have no more than about 8% loss of aprepitant, more preferably, no more than about a 5% loss of aprepitant, more preferably, no more than about a 3% loss of aprepitant under typical commercial storage conditions (i.e., 25° C./60% RH or 2-8° C.) for at least 2 months, for at least 3 months, at least 6 months, at least 9 months, at least 12 months, at least 15 months, at least 18 months, at least 21 months, at least 24 months, at least 30 months and at least 36 months.

In one embodiment, the stable emulsion compositions of the present invention are stable over a wide range of temperature, e.g., ˜20° C. to 40° C. The compositions of the present invention may be stored at about 5° C. to about 25° C.

The term “degradation product,” as used herein, refers to an unwanted chemical or impurity (including, but not limited to known or unknown related substances) that can develop during the manufacturing, transportation, and/or storage of drug products and can affect the efficacy of pharmaceutical products. It can form in response to changes in light, temperature, pH, and humidity, or due to inherent characteristics of active ingredient, such as their reaction with excipients or on contact with the packaging.

The term “parenteral” or “injectable” refers to routes selected from subcutaneous (SC), intravenous (IV), intramuscular (IM), intradermal (ID), intraperitoneal (IP) and the like.

The expression “bioequivalent” or “bioequivalence” is a term of art and is intended to be defined in accordance with Approved Drug Products with Therapeutic Equivalence Evaluations, 41st Edition, which is published by the U.S. Department of Health and Human Services, and is commonly known as the “Orange Book”. Generally, bioequivalence can be defined as the absence of significant difference in the rate and extent to which the active ingredient or active moiety in pharmaceutical equivalents or pharmaceutical alternatives becomes available at the site of drug action when administered at the same molar dose under similar conditions in an appropriately designed study. Bioequivalence of different formulations of the same drug substance involves equivalence with respect to the rate and extent of drug absorption. The pharmacokinetic characteristics of the concentration-time curve, such as the maximum observed plasma concentration (Cmax), the time to reach C max, and the area under the plasma concentration versus time curve (AUC), are examined by statistical procedures which are well-established in the field of pharmacokinetics. Two formulations whose rate and extent of absorption differ by −20%/+25% or less are generally considered to be bioequivalent.

As used herein, the term “storage” refers to the holding of a composition under controlled or uncontrolled conditions for a period ranging from a few minutes to several months or longer. Storage conditions that can be controlled include, for example, temperature, humidity, and the level of light. In many cases, storage of a pharmaceutical formulation is under industry acceptable standards and/or standards that are mandated by regulatory agencies, such as USFDA.

By “therapeutically effective” amount is meant the amount of a drug sufficient to treat, prevent, or ameliorate a condition in a subject or patient. The effective amount of aprepitant, used to practice the present invention for therapeutic management of a condition may be determined and adjusted by a person of ordinary skill to provide the appropriate amount and dosage regimen, e.g., depending upon one or more of the manners of administration, the age, body weight, sex, and/or general health of the patient.

As used herein, “prolonged duration” refers to the holding of a composition under controlled or uncontrolled conditions for a period of more than 60 days.

As used herein, “no significant loss of potency” means that no more than about a 10% loss of aprepitant under typical commercial storage conditions.

The objective of the present invention is to increase the stability of aprepitant emulsion dosage forms which can remain stable at room temperature for prolonged duration. Another objective of the present invention is to provide stable injectable emulsions of aprepitant without significant loss of potency.

The present invention further relates to methods for effectively treating or preventing nausea and vomiting induced by various events, including chemotherapy (moderately or highly emetogenic chemotherapy), radiation therapy, and surgery.

The inventive pharmaceutical compositions described herein are provided in the form of an emulsion suitable for injection. The inventive emulsion compositions can be administered either as an intravenous bolus injection over a period of about 2 minutes or as an intravenous infusion over a period of about 30 minutes. The parenterally acceptable diluents used for intravenous infusion may comprise water, water for injection, saline, half normal saline, dextrose solution (5%), alcohol, ethanol, glycerine, polyol (for example, propylene glycol, and polyethylene glycol, and the like), dimethylacetamide. N-methyl-pyrrolidone, dimethyl sulfoxide, ringer's solution, isotonic sodium chloride solution, or suitable mixtures thereof. According to the present invention, the compositions may be provided in a kit form along with a parenterally acceptable diluent.

The present invention relates to parenteral emulsions of aprepitant, particularly wherein aprepitant is present at a concentration ranging from about 2 mg/mL to about 20 mg/mL, preferably about 7.2 mg/mL.

Preferably, the stable pharmaceutical compositions for human use will be provided as an emulsion which is suitable for intravenous administration. The pharmaceutical compositions of the invention may be formulated according to conventional pharmaceutical practice and can be administered in any conventional manner. It will be readily appreciated by those skilled in the art how to administer compositions of the present invention to a human subject.

In an embodiment, a stable emulsion suitable for parenteral administration is provided comprising an oil phase, wherein the oil phase comprises aprepitant, an emulsifier, a co-emulsifier and an oil; and an aqueous phase, wherein the aqueous phase comprises water, a tonicity adjuster and buffering agent. The active ingredient, aprepitant, is present in the oil phase with an emulsifier, a co-emulsifier and an oil. The oil phase is then combined with an aqueous phase comprising water, tonicity adjuster and a buffering agent to generate a stable emulsion.

The inventive composition is in the form of an oil-in-water emulsion which remains stable over prolonged period of time and is suitable for intravenous infusion and bolus administration.

### A. Oil Phase

In an embodiment, a stable emulsion suitable for parenteral administration is provided comprising an oil phase, wherein the oil phase comprises aprepitant, an emulsifier, a co-emulsifier and an oil.

The term “emulsifier” refers to compounds which stabilize the composition by reducing the interfacial tension between the oil phase and the aqueous phase and is a compound that typically comprise at least one hydrophobic group and at least one hydrophilic group. These emulsifiers (which may also be referred to as surfactants) are preferably used in amounts effective to provide, optionally together with further surfactants present, a stable and even distribution of the oil phase within the aqueous phase. In particular, the emulsifier is selected from the group of emulsifiers that have been approved for parenteral administration. Preferably, the emulsions comprise lecithin as emulsifier, more preferably the lecithin is selected from the group consisting of egg lecithin, soy lecithin, and mixtures thereof. The term “lecithin” includes a complex mixture of acetone-insoluble phosphatides, of which phosphatidylcholine is a significant component. The term lecithin is also used as a synonym for phosphatidylcholine. Useful lecithin includes, but are not limited to, egg yolk-, egg-, soybean-, and com-derived lecithin. In one embodiment, the emulsifier is lecithin, such as egg yolk-derived lecithin. The terms egg lecithin and egg yolk derived lecithin are used interchangeable throughout. The compositions described herein preferably comprise lecithin as an emulsifier.

In one embodiment, the composition comprises about 10 wt/wt % to about 25 wt/wt %, about 12 wt/wt % to about 22 wt/wt %, about 15 wt/wt % to about 20 wt/wt %, about 17 wt/wt % to about 18 wt/wt %, about 16 w/wt % to about 19 wt/wt %, of emulsifier. In another embodiment, the composition comprises about about 15 wt/wt %, about 16 wt/wt %, about 17 wt/wt %, about 18 wt/wt %, about 19 wt/wt %, about 20 wt/wt %, about 21 wt/wt %, about 22 wt/wt %, about 23 wt/wt %, about 24 wt/wt % or about 25 wt/wt % of emulsifier. In another aspect, the emulsifier is lecithin. In yet another aspect, the lecithin is egg lecithin.

In an aspect, the ratio of emulsifier to aprepitant (wt %:wt %) in the composition ranges from about 15:1 to about 40:1, about 20:1 to about 30:1, about 23:1 to about 27:1 or about 22:1 to about 27:1. In another aspect, the ratio of emulsifier to aprepitant (wt %:wt %) in the composition is about 20:1, about 21:1, about 22:1, about 23:1, about 24:1, about 25:1, about 26:1, about 27:1, about 28:1, about 29:1 or about 30:1.

In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) in the composition ranges from 23:1 to 27:1, wherein the emulsifier concentration in the said composition ranges from 16 wt/wt % to 19 wt/wt %.

In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) ranges from 23:1 to 27:1, wherein the emulsifier concentration in the said composition ranges from 16 wt/wt % to 19 wt/wt %.

In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein emulsifier is substantially present in the oil phase.

In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein preferably there is no emulsifier in the aqueous phase.

The emulsion according to the present invention further comprises a pharmaceutically acceptable co-emulsifier.

The term co-emulsifier refers to molecules that further increase the stability of the emulsion. In addition to making the environment more hydrophobic by reducing the dielectric constant of water, co-emulsifiers increase the solubilization of hydrophobic molecules. The co-emulsifier is selected from the group consisting of alcohol, dehydrated alcohol, ethanol, propylene glycol, polyethylene glycol or mixtures thereof. In another embodiment the co-emulsifier is dehydrated alcohol. In a more preferred embodiment, the co-emulsifier is ethanol. Overages of ethanol will be used considering the evaporative losses during manufacturing. In a more preferred embodiment, not more than about 30% overages, not more than about 25% overages, not more than about 20% overages, not more than about 15% overages, not more than about 10% overages, not more than about 5% overages, not more than about 3% overages, are used in the inventive emulsion formulation. In yet another embodiment, the emulsion formulation, may not contain overages for ethanol.

In one embodiment, the emulsion comprises a co-emulsifier in an amount of about 1 wt/wt % to about 10 wt/wt %, about 2.5 wt/wt % to about 7.5 wt/wt %, or about 4 wt/wt % to about 6 wt/wt %. In another embodiment, the composition comprises less than about 10 wt/wt %, less than about 9 wt/wt %, less than about 8 wt/wt %, less than about 7 wt/wt %, less than about 6 wt/wt %, less than about 5 wt/wt %, less than about 4 wt/wt %, less than about 3 wt/wt %, less than about 2 wt/wt % or less than about 1 wt/wt % of co-emulsifier.

Further, the oil phase comprises an oil. Such oils are selected from the group consisting of structurally modified or hydrolyzed coconut oil, olive oil, soybean oil, safflower oil, triglycerides (long chain, medium chain and short chain triglycerides are all included within the scope of the term ‘triglycerides’), octyl and decyl glycerate, ethyl oleate, glyceryl linoleate, ethyl linoleate, glyceryl oleate, cholesteryl oleate/linoleate or a mixture thereof. In one embodiment, the composition comprises about 1 wt/wt % to about 30 wt/wt %, about 5 wt/wt % to about 20 wt/wt %, about 7 wt/wt % to 1 about 5 wt/wt % or about 8 wt/wt % to about 10 wt/wt % of an oil. In an embodiment, the oil is soybean oil.

In one aspect, a pharmaceutical composition suitable for intravenous administration is provided which comprises a stable emulsion comprising an oil phase, wherein the oil phase comprises aprepitant, an emulsifier, a co-emulsifier and an oil; and an aqueous phase, wherein the aqueous phase comprises water, a tonicity agent and a pH-adjusting agent.

In one embodiment, the composition is an oil-in-water emulsion wherein the oil is selected from the group consisting of structurally modified or hydrolyzed coconut oil, olive oil, soybean oil, safflower oil, triglycerides, octyl and decyl glycerate, ethyl oleate, glyceryl linoleate, ethyl linoleate, glyceryl oleate, cholesteryl oleate/linoleate or a mixture thereof. In one embodiment, the composition comprises about 5 wt/wt % (weight/weight %) to about 15 wt/wt %, about 5 wt/wt % to about 10 wt/wt %, about 7 wt/wt % to about 10 wt/wt % or about 8 wt/wt % to about 9 wt/wt % oil, in another embodiment, the oil is soybean oil.

In one embodiment, the ratio of oil to aprepitant (wt %:wt %) in the composition ranges from about 11:1 to 20:1, about 11:1 to about 15:1, about 12:1 to about 16:1, 12:1 to about 14:1, about 11:1 to about 15:1, about 12:1 to 1 about 4:1, about 12.5:1 to about 13.5:1, about 13:1 to about 14:1, or about 12:1 to about 15:1. In another embodiment, the ratio of oil to aprepitant (wt %:wt %) in the composition is about 11:1 to 20:1, about 11:1 to about 15:1, about 12:1 to about 16:1, about 12:1 to about 14:1, about 11:1, about 11.5:1, about 12:1, about 12.5:1, about 13:1, about 13.5:1, about 14:1, about 14.5:1 or about 15:1, about 15.5:1, or about 16:1.

In one aspect, the ratio of emulsifier to oil (wt %:wt %) in the composition ranges from about 0.5:1 to about 4:1, about 1:1 to about 3:1, or about 1.5:1 to about 2.5:1. In another embodiment, the ratio of emulsifier to oil (wt %:wt %) in the composition is about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.2:1, about 2.4:1, about 1.05:1, about 1.15:1, about 1.25:1, about 1.35:1, about 1.45:1, about 1.55:1, about 1.65:1, about 1.75:1, about 1.85:1, about 1.90:1, about 1.95:1.

### B. Aqueous Phase

The aqueous phase of the aprepitant emulsion is a mixture of water and other pharmaceutically acceptable excipients selected from tonicity adjuster, buffering agent, pH-adjusting agent, osmotic agents and antioxidants.

The emulsion may comprise at least one pharmaceutically acceptable tonicity adjuster. Tonicity adjusters are used to confer tonicity. Suitable tonicity adjusters may be selected from the group consisting of sodium chloride, mannitol, lactose, dextrose, sorbitol, xylitol, glucose, trehalose, maltose, raffinose, sucrose, glycerol or mixtures thereof. In one embodiment, the composition comprises about 1 wt/wt % to about 25 wt/wt %, about 2 wt/wt % to about 20 wt/wt %, about 3 wt/wt % to about 15 wt/wt %, or about 3 wt/wt % to about 8 wt/wt % tonicity adjuster. In another embodiment, the composition comprises about 1 wt/wt %, about 2 wt/wt %, about 3 wt/wt %, about 4 wt/wt %, about 5 wt % wt %, about 6 wt/wt %, about 7 wt/wt %, about 8 wt/wt %, about 9 wt/wt %, or about 10 wt/wt %, about 11 wt/wt %, about 12 wt/wt %, about 13 wt/wt %, about 14 wt/wt %, about 15 wt/wt % of tonicity adjuster. In a preferred embodiment, the tonicity adjuster is sucrose.

In still another embodiment, the composition comprises no tonicity adjuster.

The aqueous phase further contains a buffering agent to promote stability of the emulsion formulation. The drug substance may degrade; for example, lipophilic drugs will partition into the oil phase, which will confer some degree of protection, but hydrolytic degradation may still occur at the oil-water interface. Possible chemical degradation within parenteral fat emulsions includes oxidation of unsaturated fatty acid residues present in oils and lecithin, and hydrolysis of phospholipids (present in lecithins) leading to the formation of free fatty acids (FFA) and lysophospholipids. Such degradants lower pH, which may then promote further degradation. Thus, pH should be controlled during manufacture and emulsion formulations may include a buffering agent to provide additional control. Any decrease in pH over the assigned shelf-life may be indicative of chemical degradation. Suitable buffers are well known to the person skilled in the art and include, but are not limited to, phosphate buffer, citrate buffer, tris buffer, carbonate buffer, succinate buffer, maleate buffer and borate buffer, sodium hydroxide, potassium hydroxide, magnesium hydroxide, sodium carbonate, sodium linoleate, sodium oleate, potassium carbonate, potassium linoleate, potassium oleate, and mixtures thereof. In another embodiment, the buffering agent is sodium oleate.

In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein buffering agent is present only in the aqueous phase at a concentration of about 0.6% wt/wt.

In one embodiment, the composition has a pH of about 6 to about 12, about 6 to about 11, about 6 to about 10, about 6 to about 9, about 7 to about 9, about 7 to about 11, about 7.5 to about 11, about 7.5 to about 8.7, or about 7.5 to about 9.

In an embodiment, the composition is a stable fine emulsion maintaining an intensity-weighted mean particle size as determined by dynamic light scattering (DLS) of about 30 nm to about 250 nm. In another embodiment, the average droplet size is maintained below about 150 nm for a period of at least 1 month, 3 months, 6 months, 9 months, 12 months, 2 years or 3 years at 5° C. or at room temperature.

In one embodiment, the ratio of emulsifier and buffering agent to aprepitant (wt %:wt %) in the composition ranges from about 20:1 to about 35:1, about 22:1 to about 30:1, about 24:1 to about 28:1 or about 25:1 to about 26:1. In another embodiment, the ratio of emulsifier to buffering agent (wt %:wt %), may range from 28:1 to about 33:1, about 29:1 to about 32:1, or about 29:1 to about 31:1. In another embodiment, the ratio of emulsifier to buffering agent (wt %:wt %) in the composition is about 28:1, about 29:1, about 30:1, about 31:1, about 32:1 or about 33:1, more preferably at about 30:1. In one embodiment, the ratio of buffering agent to aprepitant (wt %:wt %) in the composition ranges from about 0.1:1 to about 2.0:1, about 0.4:1 to about 1.5:1 or about 0.8:1 to about 1:1.

In one embodiment, the inventive composition may further optionally contain an osmotic agent selected from the group consisting of glycerol, sorbitol, xylitol, mannitol, glucose, trehalose, maltose, sucrose, raffinose, lactose, dextran, polyethylene glycol, or propylene glycol.

In yet another embodiment, the inventive composition may further optionally contain a pH adjusting agent. Suitable pH adjusting agents include, but are not limited to, sodium hydroxide, potassium hydroxide, magnesium hydroxide, sodium carbonate, tris, sodium linoleate, sodium oleate, potassium carbonate, potassium linoleate, potassium oleate, and mixtures thereof.

### C. Preparation of the Emulsion

The present invention relates to a method for preparing an aprepitant emulsion for parenteral administration, wherein the method comprises a) preparing an oil phase comprising aprepitant, an emulsifier, co-emulsifier and an oil b) preparing an aqueous phase comprising water for injection, tonicity adjuster, buffering agents and optionally an agent for pH adjustment and/or an osmotic agent, c) forming a coarse emulsion by mixing the oil phase provided in step a) with the aqueous phase provided in step b) using high speed homogenization; d) forming a fine emulsion by high-pressure homogenizing the coarse emulsion obtained in step c) and e) sterilizing the emulsion obtained in step d).

### Step a)

Emulsifier and co-emulsifier are added and mixed together in a sequential manner at a temperature of about 55° C.±5° C. To the above mixture, is added a weighed quantity of aprepitant under continuous stirring. An oil is added to the above mixture with stirring until a clear yellow solution is obtained. The oil phase is prepared at a temperature of about 25′ C. to about 80° C., about 40° C. to about 70° C., about 50° C. to about 60° C., or at about 25° C., about 35° C., about 45° C., about 50° C., about 55° C., about 60° C., about 65° C., about 70° C. or about 75′ C, preferably at about 55° C. The dissolution of emulsifier in co-emulsifier is performed in a step-wise manner.

### Step b)

The aqueous phase is prepared by dissolving the tonicity adjuster and buffering agent in water for injection. The aqueous phase may optionally contain an osmotic agent or a pH adjusting agent. The aqueous phase is prepared at a temperature of about 25° C. to about 80° C., about 40° C. to about 70° C., about 50° C. to about 60° C., or at about 25° C., about 35° C., about 45° C., about 50° C., about 55° C., about 60° C., about 65° C., about 70° C. or about 75° C., preferably at about 55° C.

### Step c)

The oil phase provided in step a) is mixed with the aqueous phase provided in step b) thereby forming a coarse emulsion. The mixing may be carried out by any method known to those skilled in the art. Preferably, the mixing is carried out using a high-speed homogenizer.

The high-speed homogenization is performed at a speed of about 2,000 rpm (revolutions per minute) to about 25,000 rpm. The high-speed homogenization is preferably performed at a speed of about 11.000±1000 rpm. The high-speed homogenization is performed for a time period of about 0.5 min to about 2 hours, about 1 min to about 1.5 hours min, or about 1 min to about 1 hour. The high-speed homogenization is preferably performed for a time period of about 20 to about 40 min or for about 30 min. The high-speed homogenization is performed at about 20° C. to about 80° C., about 30° C. to about 70° C., about 40° C. to about 60° C., or about 50° C. to about 60° C. The high-speed homogenization is performed at about 30° C., about 35° C., about 40° C., about 45° C., about 50° C., about 55° C., about 60° C., about 65° C. or about 70° C., preferably at about 55° C.

### Step d)

The coarse emulsion of step c) is further homogenized in order to provide a fine emulsion. This homogenization may be carried out by any suitable method known to those skilled in the art. Preferably, the homogenization is carried out using a high-pressure homogenizer or a microfluidizer.

The high-pressure homogenization is performed at a pressure of about 10,000 psi (pounds per square inch) to about 30,000 psi. The high-pressure homogenization is preferably performed at a pressure of about 15,000 psi. The high-pressure homogenization is performed with cooling. The high-pressure homogenization is performed with cooling which is sufficient to bring the temperature of the emulsion at the outlet of the process to about 0° C. to about 60° C., about 10° C. to about 40° C., about 20° C. to about 30° C., or to about 20° C., about 25° C. or about 30° C. These high-pressure homogenization cycles may be repeated to sufficiently to reduce oil droplet size. The number of cycles/passes may be from about 2 to about 20, about 5 to about 13, about 8 to about 12, about 10 to about 11.

### Step e)

The fine emulsion obtained in step d) is sterilized to ensure its suitability for parenteral administration. The sterilization may be carried out by any suitable method known to those skilled in the art. For example, the inventive injectable emulsion may be sterilized using a technique selected from the group consisting of filtration through an aseptic filtration-filling-sealing process, terminal sterilization, autoclaving, incorporation of sterilizing agents, irradiation, and heating. In particular, the sterilization is carried out by filtering the fine emulsion through a membrane filter. The filter can be a Nylon 6.6 membrane filter, polyethersulfone (PES) membrane filter, polyvinylidene difluoride (PVDF) membrane filter, polytetrafluoroethylene (PTFE) membrane filter, or combinations of two or more filters. In yet another embodiment, the filter has a pore size of about 0.2 μm (micrometers).

In an embodiment, the process of preparation of a stable injectable emulsion composition of aprepitant is provided, wherein the process comprises; (a) combining the emulsifier and the co-emulsifier to provide a first mixture, adding aprepitant to the first mixture to provide a second mixture, and adding the oil to the second mixture to provide an oil phase; (b) combining water, the tonicity adjuster, and the buffering agent to provide an aqueous phase; (c) homogenizing the oil phase with the aqueous phase to generate the emulsion; and (d) sterilizing the emulsion, wherein the oil phase and aqueous phase are formed at a temperature of about 55° C.

In an embodiment, the present invention provides a stable injectable emulsion comprising (a) an oil phase comprising aprepitant, an emulsifier, co-emulsifier and an oil and (b) an aqueous phase comprising tonicity adjuster and buffering agent.

In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) in the composition ranges from 23:1 to 27:1, wherein the emulsifier concentration in the said composition ranges from 16 wt/wt % to 19 wt/wt %, and wherein the composition remains stable at 25° C./60% RH for at least 6 months.

In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) ranges from 23:1 to 27:1, wherein the emulsifier concentration in the composition ranges from 16 wt/wt % to 19 wt/wt %, and wherein the composition remains stable at 25° C./60% RH for at least 6 months.

In an embodiment, a stable injectable emulsion composition of aprepitant is provided consisting of (i) an oil phase, which comprises aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) in the composition ranges from 23:1 to 27:1, wherein the emulsifier concentration in the said composition ranges from 16 wt/wt % to 19 wt/wt %, and wherein the composition remains stable at 25° C./60% RH for at least 6 months.

In an embodiment, a stable injectable emulsion composition of aprepitant is provided consisting of (i) an oil phase, which comprises aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) ranges from 23:1 to 27:1, wherein the emulsifier concentration in the said composition ranges from 16 wt/wt % to 19 wt/wt %, and wherein the composition remains stable at 25° C./60% RH for at least 6 months.

In an embodiment, a stable injectable emulsion composition of aprepitant is provided consisting of (i) an oil phase, which consists of aprepitant, an emulsifier, a co-emulsifier and an oil; and (ii) an aqueous phase, which consists of water, a tonicity adjuster and a buffering agent, wherein the ratio of emulsifier to aprepitant (wt %:wt %) in the composition ranges from 23.1 to 27:1, wherein the emulsifier concentration in the said composition ranges from 16 wt/wt % to 19% wt/wt, and wherein the composition remains stable at 25° C./60% RH for at least 6 months.

In an embodiment, a stable injectable emulsion composition of aprepitant is provided consisting of (i) an oil phase, which consists of aprepitant, an emulsifier, a co-emulsifier and an oil; and (ii) an aqueous phase, which consists of water, a tonicity adjuster and a buffering agent, wherein the ratio of emulsifier to aprepitant (wt %:wt %) ranges from 23:1 to 27:1, wherein the emulsifier concentration in the said composition ranges from 16 wt/wt % to 19% wt/wt, and wherein the composition remains stable at 25° C./60% RH for at least 6 months.

In an embodiment, a stable injectable emulsion composition of aprepitant is provided consisting of (i) an oil phase, which consists of aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) in the composition ranges from 23:1 to 27:1, wherein the emulsifier concentration in the said composition ranges from 16 wt/wt % to 19 wt/wt %, and wherein the composition remains stable at 25° C./60% RH for at least 6 months.

In an embodiment, a stable injectable emulsion composition of aprepitant is provided consisting of (i) an oil phase, which consists of aprepitant, an emulsifier, a co-emulsifier and an oil; and (ii) an aqueous phase, which comprises water, a tonicity adjuster and a buffering agent, wherein the ratio of emulsifier to aprepitant (wt %:wt %) ranges from 23:1 to 27:1, wherein the emulsifier concentration in the said composition ranges from 16 wt/wt % to 19 wt/wt %, and wherein the composition remains stable at 25° C./60% RH for at least 6 months.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the emulsion has an osmolality value of between about 100 mOsm/kg and about 600 mOsm/kg.

The emulsion has an osmolality in the range of about 100 to about 600 mOsmol/kg, about 150 to about 500 mOsmol/kg, about 200 to about 400 mOsmol/kg. The osmolality of the inventive emulsion can be about 100 mOsmol/kg, about 120 mOsmol/kg, about 140 mOsmol/kg, about 160 mOsmol/kg, about 180 mOsmol/kg, about 200 mOsmol/kg, about 220 mOsmol/kg, about 240 mOsmol/kg, about 260 mOsmol/kg, about 280 mOsmol/kg, about 300 mOsmol/kg, about 320 mOsmol/kg, about 340 mOsmol/kg, about 360 mOsmol/kg, about 380 mOsmol/kg, about 400 mOsmol/kg, about 420 mOsmol/kg, about 440 mOsmol/kg, about 460 mOsmol/kg, about 480 mOsmol/kg, about 500 mOsmol/kg, about 520 mOsmol/kg, about 540 mOsmol/kg, about 550 mOsmol/kg, about 580 mOsmol/kg or about 600 mOsmol/kg.

In an embodiment, the present invention provides stable injectable emulsion comprising (a) an oil phase comprising aprepitant, an emulsifier, a co-emulsifier and an oil and (b) an aqueous phase comprising a tonicity adjuster and a buffering agent, wherein the emulsion has a viscosity ranging from about 1 cP (centipoise) to about 50 cP, about 10 cP to about 30 cP, or about 10 cP to about 35 cP.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the emulsion has a globule size (Z average) of about 10 nm to about 200 nm, about 20 nm to about 180 nm, about 40 nm to about 150 nm, preferably about 50 nm to about 120 nm.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the emulsion has a zeta potential ranging from about −100 mV to about −200 mV, about −15 mV to about −150 mV, about −20 mV to about −100 mV, about −25 mV to about −50 mV, or about −25 mV to about −40 mV.

Stability: In an embodiment, a stable injectable emulsion composition of aprepitant is provided comprising (i) an oil phase, which comprises aprepitant, an emulsifier, a co-emulsifier and an oil; and (ii) an aqueous phase, which comprises water, a tonicity adjuster and a buffering agent, wherein the composition remains free of visible aprepitant crystals and wherein there is no evidence of phase separation when stored for at least 6 months at 2-8° C. and at 25° C./60% RH.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the emulsion is stable when stored at room temperature conditions for prolonged duration without significant loss of potency.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the emulsion is stable for at least 2 months, or for at least 3 months, for at least 6 months, for at least 9 months, for at least 12 months, for at least 24 months, for at least 30 months, or for at least 36 months, when stored at 25° C./60% RH or at 2° C. to 8° C. or at room temperature.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the emulsion is physically and chemically stable for at least 2 months, or for at least 3 months, for at least 6 months, for at least 9 months, for at least 12 months, for at least 24 months, for at least 30 months, or for at least 36 months, when stored at 25° C./60% RH or at 2° C. to 8° C. or at room temperature.

In an embodiment, a stable emulsion suitable for parenteral administration is provided comprising (i) an oil phase comprising (a) aprepitant, (b) egg lecithin, (c) ethanol, and (d) soybean oil; and (ii) an aqueous phase comprising (a) water, (b) sucrose, and (c) sodium oleate; wherein the ratio of egg lecithin to aprepitant (wt %:wt %) in the composition is about 25:1; wherein the egg lecithin concentration in the composition is about 18 wt/wt %; wherein the composition is an emulsion; wherein the ratio of the oil phase to the aqueous phase (wt %:wt %) in the composition is about 30:70; and wherein the composition remains stable at 25° C./60% RH for at least 6 months.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the level of total impurity is less than about 5.0% (w/w), less than about 4.0% (w/w), less than about 3.0% (w/w), less than about 2.0% (w/w), less than about 1.0% (w/w), less than about 0.5% (w/w), as measured by HPLC, when stored for at least 2 months, for at least 3 months, for at least 6 months, for at least 9 months, for at least 12 months, or for at least 24 months, when stored at 25° C./60% RH or at 2° C. to 8° C. or at room temperature.

A known impurity is the one for which structural characterisation has been achieved. An unknown impurity is the one for which a structural characterisation has not been achieved and that is defined solely by qualitative analytical properties (for example, relative retention time). In particular, the following known impurities were monitored in the stable aprepitant injectable emulsion composition of the present invention.

TABLE NO. 1
Details of known Impurities

| S. No | Impurity | IUPAC Name |
|---|---|---|
| 1 | Desfluoro | 5-[[(2(R),3S)-2-[(R)-1-[3,5-Bis(trifluoromethyl) |
| | aprepitant | phenyl]ethoxy]-3-phenylmorpholino]methyl]-2H-1,2,4- |
| | | triazol-3(4H)-one |
| 2 | Impurity A | 5-[[2(R)-[1(S)-(3,5-bis(trifluoromethyl) phenyl]ethoxy]- |
| | | 3(S)-(4-fluorophenyl)-4-morpholinyl] methyl]-1,2- |
| | | dihydro-3H-1,2,4-triazol-3-one |
| 3 | Impurity B | Hydrazinecarboxylic acid, 2-[2-[(2R,3S)-2-[(1R)-1- |
| | | [3,5-bis(trifluoromethyl)phenyl]ethoxy]-3- |
| | | (4-fluorophenyl)-4-morpholinyl]-1-iminoethyl]-, |
| | | methyl ester |

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the level of total known impurities is less than about 5.0% (w/w), less than about 4.0% (w/w), less than about 3.0% (w/w), less than about 2.0% (w/w), less than about 1.0% (w/w), less than about 0.5% (w/w), less than about 0.4% (w/w), less than about 0.3% (w/w), less than about 0.2% (w/w), or less than about 0.1% (w/w), as measured by HPLC, when stored for at least 2 months, for at least 3 months, for at least 6 months, for at least 9 months, for at least 12 months, or for at least 24 months, when stored at 25° C./60% RH or at 2° C. to 8° C. or at room temperature.

In an embodiment, the present invention provides a stable injectable emulsion comprising (a) an oil phase comprising aprepitant, an emulsifier, a co-emulsifier and an oil and (b) an aqueous phase comprising a tonicity adjuster and a buffering agent, wherein the level of total unknown impurities is less than about 5.0% (w/w), less than about 4.0% (w/w), less than about 3.0% (w/w), less than about 2.0% (w/w), less than about 1.0% (w/w), less than about 0.5% (w/w), less than about 0.4% (w/w), less than about 0.3% (w/w), less than about 0.2% (w/w), or less than about 0.1% (w/w), as measured by HPLC, when stored for at least 2 months, for at least 3 months, for at least 6 months, for at least 9 months, for at least 12 months, or for at least 24 months, when stored at 25° C./60% RH or at 2° C. to 8° C. or at room temperature.

A pharmaceutically inert gas may be bubbled into the emulsion to drive out oxygen. For example, the inert gas may be selected from nitrogen or carbon dioxide. Preferably, the emulsion is kept under nitrogen or carbon dioxide sparging until the concentration of dissolved oxygen is less than about 10 mg/L in the final emulsion.

Containers suitable for packaging the emulsion according to the present invention are those known in the art and include, but are not limited to, vials, cartridges, pre-filled syringes, auto-injectors, infusion bags, bottles and ampoule presentations. Containers may be fabricated from glass or from polymeric materials. Suitable containers should be of a size sufficient to hold one or more doses of aprepitant or palonosetron.

The present invention provides for stable injectable emulsions in single-dose and/or multi-dose containers. In some embodiments, the emulsion may be contained in vials or pre-filled syringes. In some embodiments, the vials may be made from clear glass, amber glass, or plastic. In some embodiments, the vials or pre-filled syringes may have a capacity in the range of about 0.1 mL to about 100 mL in volume, about 1 mL to about 50 mL, about 4 mL to about 20 mL. In some embodiments, the composition may exist in a single dose vial having a volume of about 4 mL, about 44 mL, about 10 mL, about 15 mL, about 18 mL, about 20 mL or about 25 mL.

The polymeric materials which may be used for the pre-filled syringe include, but are not limited to: polysulfone, polycarbonate, polypropylene, polyethylene ((low-density polyethylene or high-density polyethylene), ethylene/propylene copolymers, polyolefins, acrylic-imide copolymers, polyester (e.g., polyethylene terephthalate, polyethylene naphthalate and the like), Teflon™, Nylon, acetal (Delrin), polymethylpentene, polyvinylidene chloride, ethylvinylacetate, AN-copolymer etc. In addition, cyclic olefin copolymer (COC), cyclic olefin polymer (COP), crystal zenith (CZ) resin containers and similar resins can be used as rigid containers and syringes.

### Aprepitant and Palonosetron Emulsion.

The present invention further relates to stable injectable emulsions comprising aprepitant and an 5-HT3 antagonist.

5-HT3 antagonists for use in the invention includes palonosetron, ondansetron, dolasetron, tropisetron, and granisetron, and their pharmaceutically acceptable salts thereof. A preferred 5-HT3 antagonist is palonosetron, especially its hydrochloride salt.

Palonosetron is considered to be more potent in comparison to most of the existing 5HT3 antagonists and it has a half-life of 40 hours, which effectively combats delayed onset nausea or vomiting. Aprepitant in combination with such potent 5-HT3 antagonists, is believed to provide more effective and improved way of combating nausea and vomiting emanating from emetogenic medical procedures, including chemotherapy (CINV), surgery (PONV) and radiation therapy (RINV).

The present invention relates to stable injectable emulsion comprising aprepitant and palonosetron, wherein aprepitant is present at a concentration ranging from about 2 mg/mL to about 20 mg/mL, preferably about 7.2 mg/mL; and wherein palonosetron is present at a concentration ranging from about 0.001 mg/mL to about 1 mg/mL, preferably about 0.0155 mg/mL (equivalent to palonosetron 0.0138 mg, as free base).

The inventive injectable emulsions are advantageously ready-to-use (RTU) or ready-to-dilute (RTD). The invention relates to stable ready-to-use or ready-to-dilute emulsions comprising aprepitant and palonosetron, suitable for parenteral administration.

In an embodiment, a stable emulsion suitable for parenteral administration is provided comprising (i) oil phase, which comprises aprepitant, an emulsifier, a co-emulsifier and an oil; and (ii) an aqueous phase, which comprises water, a tonicity adjuster, a buffering agent and an antioxidant, wherein palonosetron is present either in the oil phase or the aqueous phase.

In an embodiment, the ratio of oil phase to aqueous phase (wt %:wt %) in the composition ranges from about 20:80 to about 80:20, about 25:75 to about 75:25 or about 30:70 to about 70:30 or about 35:65 to about 65:35 or about 40:60 to about 60:40. In another embodiment, the ratio of oil phase to aqueous phase (wt %:wt %) in the composition is about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61 or about 40:60. In a more preferred embodiment, the ratio of oil phase to aqueous phase (wt %:wt %) in the composition is about 30:70.

In an aspect, the ratio of oil phase to aqueous phase (wt %:wt %) ranges from about 30:70 to about 60:40, about 35:65 to about 50:50, about 40:60 to about 45:55, about 33:67 to about 53:47. In another embodiment, the ratio of oil phase to aqueous phase (wt %:wt %) is about 25:75, about 30:70, about 35:75, about 40:60, about 45:55, about 50:50, about 55:45, about 60:40, about 65:35. In a more preferred embodiment, the ratio of oil phase to aqueous phase (wt %:wt %) is about 45:55.

Formulating palonosetron has not proven to be an easy task, typically due to stability issues. There remains a need for developing a palonosetron formulation having improved stability. In one embodiment, the stable emulsions include an antioxidant. Antioxidants protect the active ingredient from oxidation, which can cause degradation and loss of activity. The antioxidants useful in the present invention may be any pharmaceutically acceptable compound having antioxidant activity, for example, the antioxidant may be selected form the group consisting of sodium metasulfite, sodium bisulfite, sodium sulfite, sodium thiosulfate, sodium thiosulfate pentahydrate, sodium formaldehyde sulfoxylate, thioglycerol, thiosorbitol, thioglycolic acid, cysteine hydrochloride, n-acetyl-cysteine, citric acid, alpha-tocopherol, beta-tocopherol, gamma-tocopherol, soluble forms of vitamin E, butylated hydroxyanisole (BHA), butylated hydroxytoluene (BHT), t-butylhydroquinone (TBHQ), ethylenediaminetetraacetic acid (FDTA), monothioglycerol, propyl gallate, histidine, enzymes (such as superoxide dismutase, catalase, selenium glutathione peroxidase, phospholipid hydroperoxide and glutathione peroxidase, Coenzyme Q10), tocotrienols, carotenoids, quinones, bioflavonoids, polyphenols, bilirubin, ascorbic acid, isoascorbic acid, uric acid, metal-binding proteins, ascorbic acid palmitate and mixtures thereof.

In one embodiment, preparation of the oil phase comprises dissolving palonosetron and aprepitant in a mixture of an emulsifier and co-emulsifier. The method comprises: a) preparing an oil phase by dissolving an emulsifier in a co-emulsifier, followed by addition of aprepitant and palonosetron and adding an oil to generate an oil-based mixture; b) preparing an aqueous phase by mixing water, a tonicity adjuster, a buffering agent, and at least one antioxidant to generate an aqueous mixture; c) combining the oil-based mixture and the aqueous mixture to provide a combined mixture, and subjecting the combined mixture to high speed homogenization to generate a crude emulsion; and d) subjecting the crude emulsion to high pressure homogenization to generate a fine emulsion.

In another embodiment, preparation of the aqueous phase comprises dissolving palonosetron in water along with a tonicity adjuster, a buffering agent and an antioxidant. The method comprises: a) preparing an oil phase by dissolving an emulsifier in a co-emulsifier, followed by addition of aprepitant and adding an oil to generate an oil-based mixture; b) preparing an aqueous phase by dissolving palonosetron in water along with a tonicity adjuster, a buffering agent and at least one antioxidant to generate an aqueous mixture; c) combining the oil-based mixture and the aqueous mixture and subjecting this to high speed homogenization to generate a crude emulsion; and d) subjecting the crude emulsion to high pressure homogenization to generate a fine emulsion.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, a buffering agent and at least one antioxidant, wherein the aprepitant is present in the oil phase and palonosetron is substantially present either in the oil phase or in the aqueous phase.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, a buffering agent and at least one antioxidant, wherein aprepitant is substantially present in the oil phase and palonosetron is present either in the oil phase, in the aqueous phase, or in both phases, and wherein the emulsion has an osmolality value of between about 100 mOsm/kg and about 600 mOsm/kg.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, and wherein the emulsion has a viscosity in the range from about 1 cP (centipoise) to about 50 cP, about 10 cP to about 30 Cp, or about 10 cP to about 35 cP.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, wherein the emulsion has a globule size (Z average) of about 10 nm to about 200 nm, about about 20 nm to about 180 nm, about 40 nm to about 150 nm, preferably about 50 nm to about 120 nm.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, wherein the emulsion has a ta potential ranging from about −10 mV to about −200 mV, about −15 mV to about −150 mV, about −20 mV to about −100 mV, about −25 mV to about −50 mV, or about −25 mV to about −40 mV.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, wherein the emulsion is stable when stored at room temperature conditions for prolonged duration without significant loss of potency.

In an embodiment, a stable emulsion of aprepitant and palonosetron suitable for parenteral administration is provided comprising (i) an oil phase, which comprises an emulsifier, a co-emulsifier and an oil; and ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, wherein the emulsion is stable for at least 2 months, or for at least 3 months, for at least 6 months, for at least 9 months, for at least 12 months, for at least 24 months, for at least 30 months, or for at least 36 months, when stored at 25° C./60% RH or at 2° C. to 8° C. or at room temperature.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, wherein the emulsion is physically and chemically stable for at least 2 months, or for at least 3 months, for at least 6 months, for at least 9 months, for at least 12 months, for at least 24 months, for at least 30 months, or for at least 36 months, when stored at 25° C./60% RH or at 2° C. to 8° C. or at room temperature.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, wherein the level of total impurity is less than about 5.0% (w/w), less than about 4.0% (w/w), less than about 3.0% (w/w), less than about 2.0% (w/w), less than about 1.0% (w/w), or less than about 0.5% (w/w) as measured by HPLC, when stored for at least 2 months, for at least 3 months, for at least 6 months, for at least 9 months, for at least 12 months, or for at least 24 months, at 25° C./60% RH or at 2° C. to 8° C. or at room temperature.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, buffering agent and at least one antioxidant, wherein aprepitant is substantially present in the oil phase and palonosetron is present either in the oil phase, in the aqueous phase, or in both phases, wherein the level of known impurities is less than about 3% w/w, than about 2% w/w, less than about 1.5% w/w, less than about 1% w/w, less than about 0.5% w/w, or less than about 0.1% w/w, as measured by HPLC, when stored for at least 15 days, for at least 1 month, for at least 2 months, for at least 3 months, for at least 6 months, for at least 9 months, for at least 12 months, or for at least 24 months, at 25° C./60% RH or at 2° C. to 8° C. or at room temperature.

In an embodiment, a stable emulsion of aprepitant and palonosetron suitable for parenteral administration is provided comprising (i) an oil phase, which comprises an emulsifier, a co-emulsifier and an oil; and (ii) an aqueous phase, which comprises water, a tonicity adjuster, a buffering agent and at least one antioxidant, wherein aprepitant is substantially present in the oil phase and palonosetron is present either in the oil phase, in the aqueous phase, or in both phases, wherein the level of unknown impurities is less than about 3% w/w, than about 2% w/w, less than about 1.5% w/w, less than about 1% w/w, less than about 0.5% w/w, or less than about 0.1% w/w, as measured by HPLC, when stored for at least 15 days, for at least 1 month, for at least 2 months, for at least 3 months, for at least 6 months, for at least 9 months, for at least 12 months, or for at least 24 months, at 25° C./60% RH or at 2° C. to 8° C. or at room temperature.

In an embodiment, the process of preparation of a stable injectable emulsion composition of aprepitant and palonosetron is provided, wherein the process comprises; (a) combining the emulsifier and the co-emulsifier to provide a first mixture, adding aprepitant to the first mixture to provide a second mixture, and adding the oil to the second mixture to provide an oil phase; (b) combining water, the tonicity adjuster, buffering agent and palonosetron hydrochloride to provide an aqueous phase; (c) homogenizing the oil phase with the aqueous phase to generate the emulsion; and (d) sterilizing the emulsion, wherein the oil phase and aqueous phase are formed at a temperature of about 55° C.

In an embodiment, the process of preparation of a stable injectable emulsion composition of aprepitant and palonosetron is provided, wherein the process comprises; (a) combining the emulsifier and the co-emulsifier to provide a first mixture, adding aprepitant and palonosetron to the first mixture to provide a second mixture, and adding the oil to the second mixture to provide an oil phase; (b) combining water, the tonicity adjuster, and the buffering agent to provide an aqueous phase; (c) homogenizing the oil phase with the aqueous phase to generate the emulsion; and (d) sterilizing the emulsion, wherein the oil phase and aqueous phase are formed at a temperature of about 55° C.

### Medical Use

The terms “treating” and “treatment,” when used herein, refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The invention further provides a method of treating emesis comprising parenterally administering to a patient suffering from emesis, or at risk for suffering emesis, a dosage form of the present invention. In still further embodiments, the invention provides methods of treating emesis by administering one or more of the dosage forms described herein. The dosage form is preferably administered shortly before the emesis inducing event (i.e. no more than about 2 hours before the event). The emesis may be acute phase emesis (i.e. emesis experienced within about 24 hours of an emesis inducing event), or delayed emesis (i.e. emesis experienced after the acute phase, but within about seven, about six, about five or about four days of an emesis inducing event). The emesis may constitute chemotherapy induced nausea and vomiting (“CINV”), from moderately or highly emetogenic chemotherapy, radiation therapy induced nausea and vomiting (“RINV”), or post-operative nausea and vomiting (“PONV”). Acute emesis refers to first twenty-four-hour period following emesis-inducing event. Delayed emesis refers to the second, third, fourth and fifth twenty-four-hour periods following an emesis-inducing event

The methods of the present invention are all effective at treating or preventing nausea and vomiting induced by numerous events, including chemotherapy induced nausea and vomiting (“CINV”), moderately or highly emetogenic chemotherapy, radiation therapy induced nausea and vomiting (“RINV”), and post-operative nausea and vomiting (“PONV”). The method is preferably performed shortly before the emesis inducing event (i.e. no more than about 1 or about 2 hours before the event). The methods may be used to treat nausea and vomiting during the acute phase of emesis and/or during the delayed phase.

The drugs specified by the individual embodiments may be administered by any suitable dosing regimen, as is well known in the art. A preferred parenteral dose of aprepitant per day ranges from about 20 to about 400 mg, or from about 50 to about 300 mg, but is preferably about 130 mg. A preferred parenteral dose of palonosetron per day ranges from about 0.001 to about 2.0 mg, about 0.005 to about 1.0 mg, or from about 0.01 to about 0.75 mg.

### EXAMPLES

The following examples are exemplary and not intended to be limiting. The above disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

### Example 1

Preparing Aprepitant emulsions for parenteral administration with different concentrations of egg lecithin.

TABLE NO. 2

| | | Composition | | Composition | |
|---|---|---|---|---|---|
| | | A | | B | |
| Ingredients | Function | mg/mL | w/w % | mg/mL | w/w % |
| Aprepitant | Active | 7.2 | 0.712 | 7.2 | 0.712 |
| Egg lecithin | Emulsifier | 180.0 | 17.82 | 121.2 | 12.00 |
| Ethanol | Co-Emulsifier | 28.5 | 2.82 | 28.5 | 2.82 |
| Soybean Oil | Oil | 96.4 | 9.54 | 96.4 | 9.54 |
| Sodium Oleate | Buffering agent | 6.0 | 0.59 | 6.0 | 0.59 |
| Sucrose | Tonicity adjuster | 54.0 | 5.34 | 54.0 | 5.34 |
| Water for | Vehicle | 638 | 63.16 | 696.8 | 68.98 |
| Injection | | | | | |

TABLE NO. 3

| | | Composition | | Composition | |
|---|---|---|---|---|---|
| | | C | | D | |
| Ingredients | Function | mg/ml | w/w % | mg/mL | w/w % |
| Aprepitant | Active | 7.2 | 0.712 | 7.2 | 0.712 |
| Egg lecithin | Emulsifier | 141.4 | 14.00 | 202.02 | 20.00 |
| Ethanol | Co-Emulsifier | 28.5 | 2.82 | 28.5 | 2.82 |
| Soybean Oil | Oil | 96.4 | 9.54 | 96.4 | 9.54 |
| Sodium Oleate | Buffering agent | 6.0 | 0.59 | 6.0 | 0.59 |
| Sucrose | Tonicity adjuster | 54.0 | 5.34 | 54.0 | 5.34 |
| Water for | Vehicle | 676.6 | 66.98 | 616 | 60.98 |
| Injection | | | | | |

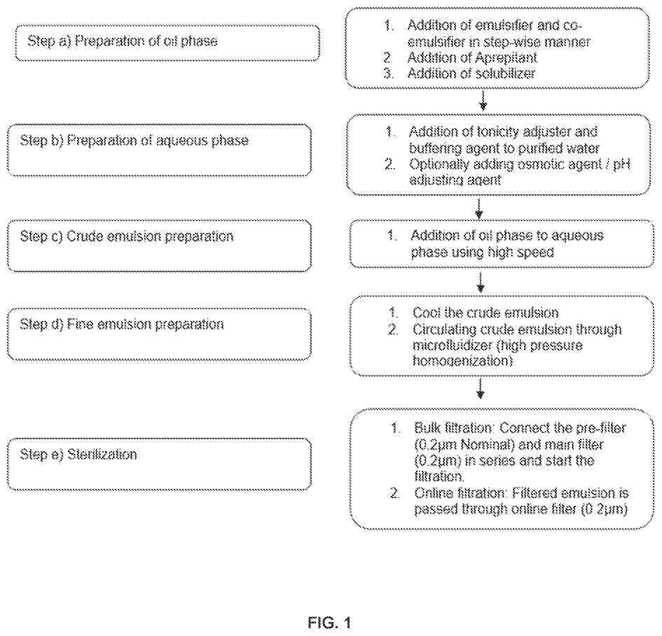

All the batches were manufactured as per the process steps described in FIG. 1.

TABLE NO 4

| | | Parameters | | |
|---|---|---|---|---|
| | | Composition A | Composition C | Composition D |
| | | Condition | | |
| | | Initial | Initial | Initial |
| | | Particulars | | |
| | | 18% | 14% | 20% |
| | | Egg Lecithin | Egg Lecithin | Egg Lecithin |
| | | Description | | |
| | | An opaque off white to amber | | |
| | | yellow colored emulsion | | |
| pH | | 8.34 | 8.32 | 8.2 |
| PFAT (>5 um) | | 0 | 0 | 0 |
| Osmolality (mOsmol/kg) | | 382 | 327 | 319 |
| Viscosity (Cp) | | 13.89 | 8.94 | 24.09 |
| Globule | Z-Average | 73.65 | 71.86 | 59.63 |
| Size Data | (nm) | | | |

| | | | |
|---|---|---|---|
| Zeta Potential (mV) | −36.8 | −36.0 | −29.4 |
| % Assay of Aprepitant | 98.9 | 98.9 | 100.4 |
| % Total Impurities | 0.04 | 0.033 | 0.055 |

An appropriate proportion of Egg Lecithin and Ethanol provides a mixture that solubilizes the aprepitant in the formulation, but 12% Egg lecithin (Composition B) and Ethanol mixture showed haziness after aprepitant addition, due to insufficient quantity of the emulsifier in the mixture.

The batch with 20%/C Egg lecithin (Composition D) was very difficult to process due to high viscosity of the oil phase when compared with 14% & 17.8% Egg lecithin batches (i.e., compositions A and C). Besides that, during the mixing stage of the oil phase to the aqueous phase, it forms a jelly-like structure which eventually impact coarse emulsion preparation.

Observation: Batches with 14% and 17.8% Egg lecithin (Composition C and A) showed manufacturing ease as well good physical chemical attributes. No phase separation was observed.

TABLE NO. 5
Stability data of Composition A

| | | Composition A | | | | |
|---|---|---|---|---|---|---|
| | | | Condition | | | |
| | | | 2-8° C. | | 25° C./60% RH | |
| | | | 6 M | 6 M | 6 M | 6 M |
| | | | Orientation | | | |
| | | Initial | Upright | Invert | Upright | Invert |
| | | Description | | | | |
| | | Opaque off white to amber colored liquid free from | | | | |
| | | visible particular matter. No phase separation | | | | |
| pH | | 8.21 | 8.07 | 8.07 | 7.48 | 7.47 |
| Osmolality (mOsm/kg) | | 311 | 312 | 301 | 302 | 304 |
| Viscosity(Cp) | | 17.11 | 18.75 | 19.47 | 26.01 | 27.45 |
| Globule Size | Z-Average (nm) | 73.59 | 76.93 | 77.99 | 105.5 | 113.2 |
| Data | Average D(10) (nm) | 45.8 | 48.3 | 49.5 | 69.6 | 76 |
| | Average D(50) (am) | 80.8 | 84.7 | 85.5 | 114 | 122 |
| | Average D(90) (nm) | 141 | 148 | 148 | 185 | 197 |
| | Average D(99) (nm) | 205 | 212 | 211 | 254 | 271 |
| | PDI | 0.154 | 0.151 | 0.147 | 0.12 | 0.121 |
| Zeta Potential (mV) | | −31.2 | −35.2 | −36.3 | −33.1 | −33.9 |
| % Assay of Aprepitant | | 99.8% | 98.4% | 98.4% | 98.00% | 98.80% |
| | Max Unknown (%) | BQL | BQL | BQL | BQL | BQL |
| | % Total Impurities | BQL | BQL | BQL | BQL | BQL |

| | | | | | |
|---|---|---|---|---|---|
| PFAT Above (5 um) | 0.00% | 0.00% | 0.00% | 0.00% | 0.00% |

### Example 2

Preparing Aprepitant emulsions for parenteral administration with different concentrations of sodium oleate.

TABLE NO 6

| | | Composition E | | Composition F | |
|---|---|---|---|---|---|
| Ingredients | Function | mg/mL | w/w % | mg/mL | w/w % |
| Aprepitant | Active | 7.2 | 0.712 | 7.2 | 0.712 |
| Egg lecithin | Emulsifier | 180.0 | 17.82 | 180.0 | 17.82 |
| Ethanol | Co-Emulsifier | 28.5 | 2.82 | 28.5 | 2.82 |
| Soybean Oil | Oil | 96.4 | 9.54 | 96.4 | 9.54 |
| Sodium Oleate | Buffering agent | 4.8 | 0.47 | 6.0 | 0.59 |
| Sucrose | Tonicity adjuster | 54.0 | 5.34 | 54.0 | 5.34 |
| Water for | Vehicle | 639.2 | 63.28 | 638 | 63.16 |
| Injection | | | | | |

All the batches were manufactured as per process steps described in FIG. 1.

TABLE NO 7

| | Condition | | |
|---|---|---|---|
| | Composition E | | |
| | | 2-8° C. | 25° C./60% RH |
| | Initial | 2 M | 1 M |
| Appearance | * | # | # |
| pH | 8.20 | 7.25 | 6.57 |
| Osmolality (mOsm/kg) | 328 | 318 | 366 |
| Viscosity (Cp) | 14.97 | 14.07 | 13.8 |
| Globule Size Data | 71.7 | 72.78 | 210.6 |
| [Z-Average (nm)] | | | |
| Zeta Potential (mV) | −29.3 | −31.7 | NP |
| % Assay of Aprepitant | 95.7 | 93.7 | 96 |
| % Total Impurities | 0.167 | NP | 0.488 |
| PFAT Above (5 μm) | 0.00% | NP | NP |

*An opaque off white to amber yellow colored emulsion
# Phase separation observed
NP = Not performed

TABLE NO 8

| | Composition F | | | | |
|---|---|---|---|---|---|
| | Condition | | | | |
| | | 2-8° C. | | 25° C./60% RH | |
| | | 6 M | | 6 M | |
| | Orientation | | | | |
| | Initial | Upright | Invert | Upright | Invert |
| Appearance | * | * | * | * | * |
| pH | 8.21 | 8.07 | 8.07 | 7.48 | 7.47 |
| Osmolality (mOsm/kg) | 311 | 312 | 301 | 302 | 304 |
| Viscosity (Cp) | 17.11 | 18.75 | 19.47 | 26.01 | 27.45 |
| Globule Size Data | 73.59 | 76.93 | 77.99 | 105.5 | 113.2 |
| [Z-Average (nm)] | | | | | |
| Zeta Potential (mV) | −31.2 | −35.2 | −36.3 | −33.1 | −33.9 |
| % Assay of Aprepitant | 99.8% | 98.4% | 98.4% | 98.00% | 98.80% |
| % Total Impurities | BQL | BQL | BQL | BQL | BQL |
| PFAT Above (5 μm) | 0.00% | 0.00% | 0.00% | 0.00% | 0.00% |

*An opaque off white to amber yellow colored emulsion

### Observation:

Composition E with 4.8 mg/mL of sodium oleate showed phase separation, with increase in average globule size beyond the acceptable limit. Composition F showed acceptable physical and chemical attributes.

### Example 3

TABLE NO 9

| Ingredients | Function | Composition G | Phase |
|---|---|---|---|
| Aprepitant | Active | 7.2 | Oil Phase |
| Egg Lecithin | emulsifier | 180.0 | |
| Ethanol | Co-emulsifier | 28.5 | |
| Soybean Oil | Oil | 96.4 | |
| Sodium Oleate | Buffering agent | 6.0 | Aqueous phase |
| Sucrose | Tonicity adjuster | 54.0 | |
| Water for Injection | Vehicle | 638 | |

TABLE NO. 10
Composition G

| | | Batch Size | | | | |
|---|---|---|---|---|---|---|
| | | | 2-8° C. | | 25° C./60% RH | |
| | | | Condition | | | |
| | | | 6 M | 6 M | 6 M | 6 M |
| | | 100 L | Orientation | | | |
| | | Initial | Upright | Invert | Upright | Invert |
| | | Description | | | | |
| | | Opaque off white to amber colored liquid free from | | | | |
| | | visible particular matter | | | | |
| pH | | 8.21 | 8.04 | 8.04 | 7.43 | 7.43 |
| Osmolality (mOsm/kg) | | 311 | 312 | 311 | 309 | 307 |
| Viscosity(Cp) | | 18.09 | 19.65 | 20.64 | 28.38 | 28.89 |
| Globule Size | Z-Average (nm) | 73.59 | 73.49 | 73.65 | 116.5 | 116.3 |
| Data | Average D(10) (am) | 45.8 | 46.6 | 46 | 77.6 | 76.9 |
| | Average D(50) (nm) | 80.8 | 79.8 | 80.3 | 125 | 125 |
| | Average D(90) (nm) | 141 | 137 | 139 | 204 | 205 |
| | Average D(99) (nm) | 205 | 193 | 199 | 281 | 282 |
| | PDI | 0.154 | 0.14 | 0.148 | 0.118 | 0,121 |
| Zeta Potential (mV) | | −31.2 | −29.9 | −30.7 | −32.6 | −33.9 |
| Assay of Aprepitant | | 99.80% | 99.1% | 100.6% | 99.5% | 98.0% |
| | Max Unknown (%) | BOL | BOL | BOL | BQL | BQL |
| | % Total Impurities | BQL | BQL | BQL | BQL | BQL |

| PFAT Above (5 um) | 0.00% | 0.00% | 0.00% | 0.00% | 0.00% |
|---|---|---|---|---|---|
| Ethanol Content (% w/w) | 99.5% | NA | | | |
| Free Drug content (μg/mL) | BLQ | BLQ | BLQ | BLQ | BLQ |
| Free fatty acid content(mmol/lit) | 49.6 | 52.33 | $2.67 | 60.19 | 58.14 |
| USP Type IV dissolution (Q point @ | 101% | NA | 98% | NA | 100% |
| 60 min) | | | | | |

Observation: Composition G showed acceptable physical and chemical attributes.

### Example 3

Preparing Aprepitant emulsions for parenteral administration with different overages of ethanol in order to minimize losses due to evaporation

Compositions G, H and I were reformulated at higher batch size using different overages of ethanol as follows.

TABLE NO 11

| Composition | G | H | I |
|---|---|---|---|
| Overages in ethanol | 30% | 10% | 5% |
| Batch size | 150 L | 100 L | 100 L |

All the batches were manufactured as per the process steps described in FIG. 1.

TABLE NO 12
Composition G

| | Batch Size | | | | |
|---|---|---|---|---|---|
| | 150 L | 2-8° C. | | 25° C./60% RH | |
| | Condition | | | | |
| | | 6 M | | 6 M | |
| | Orientation | | | | |
| | Initial | Upright | Invert | Upright | Invert |
| | Description | | | | |
| | Opaque off white to amber colored liquid | | | | |
| | free from visible particular matter | | | | |
| pH | 8.25 | 7.94 | 7.92 | 7.35 | 7.36 |
| Osmolality (mOsm/kg) | 386 | 390 | 389 | 403 | 398 |
| Viscosity (Cp) | 16.95 | 18.21 | 18.51 | 23.16 | 24.09 |
| Globule Size Data | 75.19 | 78.43 | 78.89 | 115.9 | 116.3 |
| [Z-Average (nm)] | | | | | |
| Zeta Potential (mV) | −31.1 | −37.4 | −37.9 | −34.2 | −31.1 |
| % Assay of Aprepitant | 95.2 | 96.1 | 97.5 | 94.9 | 96 |
| % Total Impurities | 0.055 | BLQ | BLQ | BLQ | BLQ |

| | |
|---|---|
| Ethanol Content (% w/w) | 125% |

TABLE NO 13
Composition H

| | Batch Size | | | | |
|---|---|---|---|---|---|
| | 100 L | 2-8° C. | | 25° C./60% RH | |
| | Condition | | | | |
| | | 6 M | | 6 M | |
| | Orientation | | | | |
| | Initial | Upright | Invert | Upright | Invert |
| | Description | | | | |
| | Opaque off white to amber colored liquid | | | | |
| | free from visible particular matter | | | | |
| pH | 8.29 | 8.04 | 8.04 | 7.43 | 7.43 |
| Osmolality (mOsm/kg) | 334 | 312 | 311 | 309 | 307 |
| Viscosity (Cp) | 18.09 | 19.65 | 20.64 | 28.38 | 28.89 |
| Globule Size Data | 77.6 | 73.49 | 73.65 | 116.5 | 116.3 |
| [Z-Average (nm)] | | | | | |
| Zeta Potential (mV) | −30.6 | −29.9 | −30.7 | −32.6 | −33.9 |
| Assay of Aprepitant | 100% | 99.1% | 100.6% | 99.5% | 98.0% |
| % Total Impurities | BQL | BQL | BQL | BQL | BQL |

| | |
|---|---|
| Ethanol Content (% w/w) | 99.5% |

TABLE NO 14
Composition I

| | Stability Condition: | | | | | |
|---|---|---|---|---|---|---|
| | | | 25° C./60% RH | | 25° C./60% RH | |
| | Orientation | | | | | |
| | | | Inverted | | Upright | |
| | Test | | | | | |
| | Initial | | 1 Month | | 1 Month | |
| | Description | | | | | |
| | Opaque, off-white to amber liquid, free from visible | | | | | |
| | particulate matter, emulsion contained in a tubular | | | | | |
| | glass vial with a red aluminium seal. | | | | | |
| Assay of Aprepitant | 96.9% | w/w | 98.0% | w/w | 97.9% | w/w |
| Content of Ethanol by GC | 100.5% w/w | | 101.7% w/w | | 103.0% w/w | |
| (in mg) | (28.6 mg) | | (29.0 mg) | | (29.3 mg) | |
| Total Impurities | 0.1% | w/w | NP | | BQL | |
| pH | 8.28 | | 7.94 | | 7.94 | |
| Viscosity | 18 | cP | 22 | cP | 24 | cP |
| Globule size distribution | 68 | nm | 78 | nm | 77 | nm |
| (Z. Average) | | | | | | |
| Zeta potential | −33 | mV | −32 | mV | −32 | mV |

| | | | |
|---|---|---|---|
| Osmolality (mOsmol/kg) | 324 | 320 | 324 |

Observation: In view of the above data, it was evident that 5% ethanol overages are adequate to compensate the losses due to handling and evaporation of ethanol.

### Example 4

Preparing Aprepitant emulsions for parenteral administration to evaluate the effect of different pH.

pH of Composition G was adjusted at pH 8.44, pH 9.56, and 11.26 pH. Hydrochloric acid and 1N Sodium Hydroxide was used for adjusting the pH of Composition G.

Observation: Samples were found to be clear at the initial and for 4 days at RT.

### Example 5

The viscosity of the product is representative of the formulation composition. The viscosity of the stable injectable emulsion composition of aprepitant of the present invention are attributed to the concentration of emulsifier (egg lecithin). The viscosity values for the composition of the present invention were observed to be higher in comparison to Cinvanti® (IV emulsion; 130 mg/18 mL).

A Syringeability study was performed to evaluate the impact of these differing viscosities of the present invention and Cinvanti® composition on Break loose and Glide force during withdrawal of the required emulsion product from the vial and while delivering the same from the syringe to the catheter. The details of study and results are provided below:

TABLE NO. 15
Syringeability study

| | | | | Pull Syringeability - | | |
|---|---|---|---|---|---|---|
| | | | | With 18G Needle | | |
| | | | | Break | Peak | Average |
| | | | | Loose | Gliding | Gliding |
| | | Vis- | Sample | Force | Force | Force |
| Batch No. | Condition | cosity | No. | (N) | (N) | (N) |
| Composition G | Initial | 19 cP | 1 | 7.384 | 28.685 | 21.88 |
| | | | 2 | 6.913 | 27.009 | 21.101 |
| | | | 3 | 7.975 | 27.286 | 21.542 |
| | | | 4 | 6.083 | 27.338 | 21.119 |
| | | | 5 | 7.212 | 30.705 | 23.551 |
| | 25° C./ | 27 cP | 1 | 9.352 | 29.448 | 23.761 |
| | 60% RH | | | | | |
| | 6 months | | 2 | 9.18 | 30.196 | 23.80S |
| Cinvanti ® | Initial | 15 cP | 1 | 6.816 | 30.211 | 23.054 |
| Batch # 84710 | | | | | | |
| Cinvanti ® | Initial | 11 cp | 1 | 14.11 | 28.61 | 23.484 |
| Batch # 84709 | | | | | | |

TABLE NO. 16

| | | | | Push Syringeability - | | |
|---|---|---|---|---|---|---|
| | | | | With 18G Needle | | |
| | | | | Break | Peak | Average |
| | | | | Loose | Gliding | Gliding |
| | | Vis- | Sample | Force | Force | Force |
| Batch No. | Condition | cosity | No. | (N) | (N) | (N) |
| Composition G | Initial | 19 cP | 1 | 6.166 | 8.59 | 5.92 |
| | | | 2 | 5.911 | 5.702 | 4.2 |
| | | | 3 | 5.44 | 6.502 | 5.111 |
| | | | 4 | 5.941 | 6.091 | 4.756 |
| | | | 5 | 4.43 | 4.115 | 3.483 |
| | 25° C./ | 27 cP | 1 | 5.44 | 7.572 | 6.14 |
| | 60% RH | | | | | |
| | 6 months | | 2 | 5.821 | 6.016 | 4.731 |
| Cinvanti ® | Initial | 15 cP | 1 | 5.395 | 4.684 | 3.919 |
| Batch # 84710 | | | | | | |
| Cinvanti ® | Initial | 11 cp | 1 | 3.494 | 3.187 | 2.627 |
| Batch # 84709 | | | | | | |

### Observation:

Based on observed results of the Break loose force and Gliding force, testing done on Composition and Cinvanti®, it can be concluded that Composition G demonstrate similar Syringeability to Cinvanti®.

### Example 5

Preparing Aprepitant and palonosetron emulsions for parenteral administration with different antioxidants.

TABLE NO. 17

| Composition | | 001 | 002 | 003 | 004 | 005 | |
|---|---|---|---|---|---|---|---|
| Ingredients | Function | mg/mL | | | | | Phase |
| Aprepitant | Active | 7.2 | 7.2 | 7.2 | 7.2 | 7.2 | Oil |
| Egg Lecithin | emulsifier | 180.0 | 180.0 | 180.0 | 180.0 | 180.0 | Phase |
| Ethanol | Co-emulsifier | 28.5 | 28.5 | 28.5 | 28.5 | 28.5 | |
| Monothioglycerol | Antioxidant | — | — | — | — | — | |
| Soybean Oil | Oil | 96.4 | 96.4 | 96.4 | 96.4 | 96.4 | |
| Sodium Oleate | Buffering | 6.0 | 6.0 | 6.0 | 6.0 | 6.0 | Aqueous |
| | agent | | | | | | phase |
| Sucrose | Tonicity | 54.0 | 54.0 | 54.0 | 54.0 | — | |
| | adjuster | | | | | | |
| EDTA | Antioxidant | 0.5 | — | — | — | 0.16 | |
| Monothioglycerol | | — | 1.3 | — | — | — | |
| Alpha-Tocopherol | | — | — | 9.0 | — | — | |
| Sodium | | — | — | — | — | — | |
| formaldehyde | | | | | | | |
| sulfoxylate | | | | | | | |
| Sodium Thiosulfate | | — | — | — | — | — | |
| Pentahydrate | | | | | | | |
| Mannitol | Tonicity | — | — | — | — | 38 | |
| | adjuster | | | | | | |
| Palonosetron HCl | Active | 0.0155 | 0.0155 | 0.0155 | 0.0155 | 0.0155 | |
| Milli-Q Water | Vehicle | 638.5 | 637.7 | 630.0 | 635.0 | 640.0 | |

TABLE NO. 18

| Composition | | 006 | 007 | 008 | 009 | 010 | 011 | 012 | |
|---|---|---|---|---|---|---|---|---|---|
| Ingredients | Function | mg/mL | | | | | | | Phase |
| Aprepitant | Active | 7.2 | 7.2 | 7.2 | 7.2 | 7.2 | 7.2 | 7.2 | Oil |
| Palonosetron HCl | Active | — | — | — | 0.0155 | — | — | — | Phase |
| Egg Lecithin | Emulsifier | 180.0 | 180.0 | 180.0 | 180.0 | 180.0 | 180.0 | 180.0 | |
| Ethanol | Co- | 28.5 | 28.5 | 28.5 | 28.5 | 28.5 | 28.5 | 28.5 | |
| | emulsifier | | | | | | | | |
| Monothioglycerol | Antioxidant | — | — | 1.3 | 1.3 | 0.3 | — | 0.65 | |
| BHT | | — | — | — | — | — | 0.002 | — | |
| Soybean Oil | Oil | 96.4 | 96.4 | 96.4 | 96.4 | 96.4 | 96.4 | 96.4 | |
| Sodium Oleate | Buffering | 6.0 | 6.0 | 6.0 | 6.0 | 6.0 | 6.0 | 6.0 | Aqueous |
| | agent | | | | | | | | phase |
| Sucrose | Tonicity | 54.0 | 54.0 | 54.0 | 54.0 | 54.0 | 54.0 | 54.0 | |
| | adjuster | | | | | | | | |
| EDTA | | — | — | — | 5 | — | 0.16 | — | |
| Monothioglycerol | Antioxidant | — | — | — | — | — | — | — | |
| Alpha-Tocopherol | | — | — | — | — | — | — | — | |
| Sodium | | 0.5 | — | — | — | — | — | — | |
| formaldehyde | | | | | | | | | |
| sulfoxylate | | | | | | | | | |
| Sodium | | — | 1.0 | — | — | — | — | — | |
| Thiosulfate | | | | | | | | | |
| Pentahydrate | | | | | | | | | |
| Mannitol | Tonicity | — | — | — | — | — | — | — | |
| | adjuster | | | | | | | | |
| Palonosetron HCl | Active | 0.0155 | 0.0155 | 0.0155 | — | 0.0155 | 0.0155 | 0.0155 | |
| Milli-Q) Water | Vehicle | 635.0 | 635.0 | 635.0 | | 635.0 | 635.0 | 638.0 | |

All the batches were manufactured as per the process steps described in FIG. 1, wherein palonosetron and antioxidants are added either in the oil phase or aqueous phase.

TABLE NO 19

| | | Composition | | |
|---|---|---|---|---|
| | | 001 | | |
| | | Condition | | |
| | | | 2-8° C. | 25° C./60% RH |
| | | Duration | | |
| | | Initial | 3 M | 2 M |
| | | Description | | |
| | | Opaque, off-white to amber liquid, | | |
| | | free from visible particulate matter | | |
| pH | | 8.07 | 7.96 | 7.73 |
| Osmolality (mOsm/kg) | | 345 | 330 | 340 |
| Viscosity (cps) | | 13.05 | 13.59 | 13.68 |
| % Assay of Palonosetron HCL | | 100.6 | 98.4 | 99.3 |
| % Assay of Aprepitant | | 97.7 | 97.2 | 97.0 |
| RS of Aprepitant | % Total Impurities | ND | BLQ | ND |
| RS of Palonosetron | Related Compound A | 0.78 | 0.73 | 1.14 |
| | (N-Oxide) | | | |
| | % Total Impurities | 0.78% | 0.73% | 1.14% |
| Globule Size Data | Z-Average (d · nm) | 74.85 | 73.01 | 113 |

| | | | |
|---|---|---|---|
| Zeta Potential (mV) | −30.8 | −31.2 | −35.6 |

TABLE NO 20

| | | Composition | | |
|---|---|---|---|---|
| | | 002 | | |
| | | Condition | | |
| | | | 2-8° C. | 25° C./60% RH |
| | | Initial | 3 M | 3 M |
| | | Description | | |
| | | Opaque, off-white to amber liquid, | | |
| | | free from visible particulate matter | | |
| pH | | 8.45 | 8.32 | 7.88 |
| Osmolality (mOsm/kg) | | 320 | 289 | 284 |
| Viscosity (cps) | | NP | NP | NP |
| % Assay of Palonosetron HCL | | 107.5 | NP | NP |
| % Assay of Aprepitant | | 103.1 | 101.9 | 100.5 |
| RS of Aprepitant | % Total Impurities | ND | BLQ | 0.077 |
| RS of Palonosetron | Related Compound A | ND | ND | ND |
| | (N-Oxide) | | | |
| | % Total Impurities | 0.60% | 1.40% | 1.62% |
| Globule Size Data | Z-Average (d · nm) | 75.9 | 75.45 | 80.65 |

| | | | |
|---|---|---|---|
| Zeta Potential (mV) | −40.6 | −40.9 | −40.5 |

TABLE NO 21

| | | Composition | | |
|---|---|---|---|---|
| | | 005 | | |
| | | Condition | | |
| | | | 2-8° C. | 25° C./60% RH |
| | | Duration | | |
| | | Initial | 1 M | 1 M |
| | | Description | | |
| | | An opaque off white to amber | | |
| | | yellow colored emulsion | | |
| pH | | 8.18 | 8.16 | 7.9 |
| Osmolality (mOsm/kg) | | 341 | 325 | 324 |
| Viscosity | | NP | NP | NP |
| % Assay of Palonosetron HCL | | 103.2 | — | — |
| % Assay of Aprepitant | | 99.0 | 98.5 | 97.4 |
| RS of Aprepitant | % Total Impurities | BLQ | ND | 0.076 |
| RS of Palonosetron | Related Compound A | 0.45 | 0.79 | 1.68 |
| | (N-Oxide) | | | |
| | % Total Impurities | 0.45 | 0.79 | 2.05 |
| Globule Size Data | Z-Average (d · nm) | 59.5 | 57.25 | 79.68 |

| | | | |
|---|---|---|---|
| Zeta Potential (mV) | −31.2 | −31.3 | −31.5 |

TABLE NO 22

Composition

| | |
|---|---|
| 006 | 007 |

Condition

| | |
|---|---|
| Initial | Initial |

Description
An opaque off white to amber
yellow colored emulsion

| pH | | 8.02 | 8.27 |
|---|---|---|---|
| Osmolality (mOsm/kg) | | 321 | 301 |
| Viscosity | | 15.51 | 15.45 |
| % Assay of Palonosetron HCL | | 101.5 | 98.0 |
| % Assay of Aprepitant | | 97.9 | 99.5 |
| RS of Aprepitant | % Total Impurities | BLQ | BLQ |
| RS of Palonosetron | Related Compound A | 0.45 | 0.36 |
| | (N-Oxide) | | |
| | % Total Impurities | 0.453 | 0.363 |
| Globule Size Data | Z-Average (d · nm) | 69.16 | 68.42 |

| | | |
|---|---|---|
| Zeta Potential (mV) | −28.7 | −32.9 |

TABLE NO 23

| | | Composition | | |
|---|---|---|---|---|
| | | 008 | 009 | 010 |
| | | Condition | | |
| | | Initial | Initial | Initial |
| | | Description | | |
| | | An opaque off white to amber | | |
| | | yellow colored emulsion | | |
| pH | | 8.24 | 8.26 | 8.15 |
| Osmolality (mOsm/kg) * | | 353 | 330 | 313 |
| Viscosity (Cp) | | 16.98 | 15.33 | NP |
| % Assay of Palonosetron HCL | | 104.0 | 100.8 | NP |
| % Assay of Aprepitant | | 96.8 | 97.3 | 96.9 |
| RS of Aprepitant** | % Total Impurities | ND | ND | ND |
| RS of Palonosetron | Related Compound A | ND | ND | 0.12 |
| | (N-Oxide) | | | |
| | % Total Impurities | ND | ND | 0.12 |
| Globule Size Data | Z-Average (d · nm) | 65.95 | 63.38 | 63.38 |

| | | | |
|---|---|---|---|
| Zeta Potential (mV) | −31.9 | −30.3 | −30.3 |

Observation: Initial properties for all the above compositions was found to be satisfactory.

TABLE NO 24

| | | Composition | | |
|---|---|---|---|---|
| | | 012 | | |
| | | Condition | | |
| | | | 25° C./60% RH- | 2-8° C.- |
| | | | Upright | Upright |
| | | Time Point | | |
| | | Initial | 06 Months | 06 Months |
| | | Description | | |
| | | | Opaque off- | Opaque |
| | | | white liquid, | amber colored |
| | | An opaque off | free from | liquid, free |
| | | white to amber | visible | from visible |
| | | yellow colored | particulate | particulate |
| | | emulsion | matter | matter |
| pH | | 8.19 | 7.44 | 7.99 |
| Osmolality (mOsm/kg) * | | 295 | 289 | 295 |
| PFAT Above (5 um) | | 0 | 0 | 0 |
| Viscosity(Cp) | | 15.55 | 34.5 | 20.5 |
| % Assay of Palanosetron HCL | | 99.3 | 98.8 | 100.3 |
| % Assay of Aprepitant | | 100.1 | 98.6 | 99.3 |
| RS of | Any unspecified | 0.021 | UA | UA |
| Aprepitant** | degradation product | | | |
| | (%) | | | |
| | % Total degradation | 0.047 | | |
| | products | | | |
| RS of | Palonosetron related | 0.123 | | |
| Palanosetron | compound A | | | |
| | Any unspecified | ND | | |
| | degradation product | | | |
| | (%) | | | |
| | % Total degradation | 0.123 | | |
| | products | | | |
| Globule | Z-Average (d · nm) | 68.78 | 100.3 | 66.18 |
| Size Data | PDI | 0.198 | 0.143 | 0.194 |
| | Average D(10) | 40.8 | 64.5 | 37.4 |
| | Average D(50) | 78.8 | 110 | 75.4 |
| | Average D(90) | 141 | 186 | 143 |

| | | | |
|---|---|---|---|
| Zeta Potential (mV) | −33.4 | −36.4 | −40.4 |
| Free Fatty Acids | 53.69 | 73.28 | 65.2 |

Observation: Composition 012 was found to be stable for at least a period of 6 months at 25° C./60% RH and at 2-8° C.

What is claimed:

1. A stable injectable composition comprising:
(i) an oil phase comprising (a) aprepitant, (b) an emulsifier, (c) a co-emulsifier, and (d) an oil; and
(ii) an aqueous phase comprising (a) water, (b) a tonicity adjuster, and (c) a buffering agent;
wherein the ratio of emulsifier to aprepitant (wt %:wt %) ranges from 23:1 to 27:1;
wherein the emulsifier concentration in the composition ranges from 16 wt/wt % to 19 wt/wt %;
wherein the composition is an emulsion;
wherein the ratio of the oil phase to the aqueous phase (wt %:wt %) in the composition ranges from about 25:75 to about 35:65; and
wherein the composition remains stable at 25° C./60% RH for at least 6 months.

2. The composition according to claim 1, wherein aprepitant is present at a concentration of about 7.2 mg/mL.

3. The composition according to claim 1, wherein emulsifier is present at a concentration of about 18 wt/wt %.

4. The composition according to claim 1, wherein the ratio of emulsifier to aprepitant (wt %:wt %) in the composition is about 25:1.

5. The composition according to claim 1, wherein the emulsifier is egg lecithin.

6. The composition according to claim 1, wherein the co-emulsifier is ethanol.

7. The composition according to claim 1, wherein emulsifier is substantially present in the oil phase.

8. The composition according to claim 1, wherein the oil is soybean oil.

9. The composition according to claim 1, wherein the tonicity adjuster is sucrose.

10. The composition according to claim 1, wherein the buffering agent is sodium oleate.

11. The composition according to claim 10, wherein sodium oleate is present in the aqueous phase at a concentration of about 0.6 wt/wt %.

12. The composition according to claim 1, wherein the composition remains free of visible aprepitant crystals and wherein there is no evidence of phase separation for at least 6 months when stored at 2-8° C. and at 25° C./60% RH.

13. The composition according to claim 1, wherein viscosity of the composition ranges from about 10 cP to about 35 cP.

14. The composition according to claim 1, wherein pH of the composition ranges from about 7.5 to about 11.0.

15. The composition according to claim 1, wherein zeta potential of the composition ranges from about −25 mV to about −40 mV.

16. A process for preparing the stable injectable composition according to claim 1 comprising:
(a) combining the emulsifier and the co-emulsifier to provide a first mixture, adding aprepitant to the first mixture to provide a second mixture, and adding the oil to the second mixture to provide an oil phase;
(b) combining water, the tonicity adjuster, and the buffering agent to provide an aqueous phase;
(c) homogenizing the oil phase with the aqueous phase to generate the emulsion; and
(d) sterilizing the emulsion,
wherein the oil phase and aqueous phase are formed at a temperature of about 55° C.

17. The composition according to claim 1, wherein
(i) the oil phase comprises (a) aprepitant, (b) egg lecithin, (c) ethanol, and (d) soybean oil; and
(ii) the aqueous phase comprises (a) water, (b) sucrose, and (c) sodium oleate;
wherein the ratio of egg lecithin to aprepitant (wt %:wt %) is about 25:1;
wherein the egg lecithin concentration in the composition is about 18 wt/wt %;
wherein the composition is an emulsion;
wherein the ratio of the oil phase to the aqueous phase (wt %:wt %) in the composition is about 30:70; and
wherein the composition remains stable at 25° C./60% RH for at least 6 months.
